SEQUENCE LISTING (26 sequences)

SEQ ID NO: 1 - protein, 186 aa, Artificial Sequence
  Hydrolase domain
KKGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLETYGYYAGFYTSLSVANNLVSAHVRNRYALWIAQWNTHCNYQGSYGLWQYSSSGSVPGVAGRVDMDYAY

SEQ ID NO: 2 - protein, 122 aa, Artificial Sequence
  Hydrolase domain
QGLKINKIVLHHNAGNLSIQDCYNVWQTREASAHYQVQSDGRIGQLVWDYDTAWHAGNWNANATSIGIEHADISTSPWQISAQCLENGAHLVAALCKLYGLGRPTWMKNVFPHSYFYATACP

SEQ ID NO: 3 - protein, 194 aa, Artificial Sequence
  Hydrolase domain
LNGIDISSYQSSINIAAVPADFVIVKATEGTGYINPCFRAHADTILNSGKLLGIYHYISGSGWQAEAEYFVNTVKDYIGRAVLALDFESGGNSAYGDTAYLQQCAQTVYNLTGVHPLIYGSQRDYGRLAAVSKATNCGLWVAQYANNNHTGYQNKPWNEDAYDCAIRQYSSSGSLPNYGGNLDLNKFYGDAAAW

SEQ ID NO: 4 - protein, 185 aa, Artificial Sequence
  Hydrolase domain
IVDVYSGSSDSIIQDPHADGVIVKATQGTSYVNPRCNHQWDLAGQLGKLRGLYHYAGGGNPESEAQYFINNIKNYVGQGILILDWESYQNSSWGDTSWPLRFVTEVHRLTGVWPLIYVQESALWQIANCAPYCGVWVAKYASMDWKSWTLPNMSVSAGAFGALTGWQFTGGDMDRSIFYLTKETW

SEQ ID NO: 5 - protein, 188 aa, Artificial Sequence
  Hydrolase domain
LKVVDVYSGSPRWYATDDNADAVIVKATQGTGYVNPFCDIDYQAAKKAGKLLGVYHYASGGDPISEANYFLKNVKGYVHEAILGLDWESAQNASWGNTNWCRQFVNEIHRQTGVWPIIYVQFSAVWQVANCADTCGLWGAGYPWYTNSWTVPPFLSSYNFAPWKDLTGWQFTGNTEDRSLFYVDANGW

SEQ ID NO: 6 - protein, 184 aa, Artificial Sequence
  Hydrolase domain
TDVFSGSADWIVTDPHAQGTIVKASQGTGYVNPKYEYQYSLAKTNGRLLGLYHYAGGNDPVAEATYFLDHIRGKVGEAVLAVDWEQYQNAAWGNPNWVRKFVDEVHRQTNVWPLIYVQESAIWQVANCANDCGLWVAKYPSMNWHSWQVPDMQVNTAPWPGYTLWQFAGDDEDRSIATVDRNGW

SEQ ID NO: 7 - protein, 43 aa, Artificial Sequence
  Cell wall binding domain
SSIDEVAREVINGAWGNGKERKQRLTQAGYDYASVQNKVNELL

SEQ ID NO: 8 - protein, 42 aa, Artificial Sequence
  Cell wall binding domain
SVDELAREVIRGAWGNGNERKQRLTQAGYDYDTVQKRVNELL

SEQ ID NO: 9 - protein, 111 aa, Artificial Sequence
  Cell wall binding domain
SEIVVDGNVSSVEIHVPWGVNNSVGMTLTRAGNVVTANGAGGIKAGDAQWAKANETIPEGFRPTSLSTITLTGGRAALLVQPDGSMYYDGDARDCTTHLSGTWITNDNQPE

SEQ ID NO: 10 - protein, 111 aa, Artificial Sequence
  Cell wall binding domain
SEITHDGDISTVYVHIPWSVQQDIRMAVVRVGNVVTVNGCGGMSAGDAQWAKANETIPEGFRPTSLSTITLTGGRAALLVQPDGSIYYDGDARDCTTHISGVWITKDTQPK

SEQ ID NO: 11 - protein, 134 aa, Artificial Sequence
  Cell wall binding domain
QFTGGDMDRSIFYLTKETWMKIANPSGSKDGWQGSGDTWQYFENGSAVKNDWRKVDNCWYYLDESGNAVTGWKQINNHWYYFDTKHDGSFGTALTGWQQINGKKYYFDLQNAWMLTGKQTIDGKLYTFGDDGAL

SEQ ID NO: 12 - protein, 62 aa, Artificial Sequence
  Cell wall binding domain
ENGKFTANTDLHLRWGATPSSSVISVLHAGDVVKYDAWARTNGFVYIRQPRGNGQYGYVAVR

SEQ ID NO: 13 - protein, 51 aa, Artificial Sequence
  Cell wall binding domain
PLTLRWGALPTSTAIAELPAGSVIDYDAWSRHDGEVWIRQPRGNGQYGYLP

SEQ ID NO: 14 - protein, 26 aa, Artificial Sequence
  Linker
VDYPSIIKNAGLNGCKNGGSDQAART

SEQ ID NO: 15 - protein, 7 aa, Artificial Sequence
  Linker
GVKAYRK

SEQ ID NO: 16 - protein, 42 aa, Artificial Sequence
  Linker
CSIRDSQNLDYMKRAGDWYDAMTGVVKQQNQTKSGEEYPMIK

SEQ ID NO: 17 - protein, 25 aa, Artificial Sequence
  Linker
QSYAKSDNQTQQSQEAQQAEPMPAI

SEQ ID NO: 18 - protein, 48 aa, Artificial Sequence
  Linker
QEATTQETKPAEAKPSIDKQIVLDDQLKAYIQSEIKAQLAKLKIKMEE

SEQ ID NO: 19 - protein, 46 aa, Artificial Sequence
  Linker
QAIAKGDGTVTHTAQPAPQKNEAKTSNVPSYAGTTWQDSLGVTWHA

SEQ ID NO: 20 - protein, 50 aa, Artificial Sequence
  Linker
QRLANPTGQVSSRPLIFGDSSASSISNNETWTDNLGDTWHREVGRFTSSR

SEQ ID NO: 21 - protein, 306 aa, Artificial Sequence
  Endolysin polypeptide
MSKKGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLETYGYYAGFYTSLSVANNLVSAHVRNRYALWIAQWNTHCNYQGSYGLWQYSSSGSVPGVAGRVDMDYAYVDYPSIIKNAGLNGCKNGGSDQAARTSSIDEVAREVINGAWGNGKERKQRLTQAGYDYASVQNKVNELLGVKAYRKSVDELAREVIRGAWGNGNERKQRLTQAGYDYDTVQKRVNELL

SEQ ID NO: 22 - protein, 298 aa, Artificial Sequence
  Endolysin polypeptide
MKNWETCDADEIKLLTTHYTKGRQGLKINKIVLHHNAGNLSIQDCYNVWQTREASAHYQVQSDGRIGQLVWDYDTAWHAGNWNANATSIGIEHADISTSPWQISAQCLENGAHLVAALCKLYGLGRPTWMKNVFPHSYFYATACPCSIRDSQNLDYMKRAGDWYDAMTGVVKQQNQTKSGEEYPMIKSEIVVDGNVSSVEIHVPWGVNNSVGMTLTRAGNVVTANGAGGIKAGDAQWAKANETIPEGFRPTSLSTITLTGGRAALLVQPDGSMYYDGDARDCTTHLSGTWITNDNQPE

SEQ ID NO: 23 - protein, 332 aa, Artificial Sequence
  Endolysin polypeptide
MALNGIDISSYQSSINIAAVPADFVIVKATEGTGYINPCFRAHADTILNSGKLLGIYHYISGSGWQAEAEYFVNTVKDYIGRAVLALDFESGGNSAYGDTAYLQQCAQTVYNLTGVHPLIYGSQRDYGRLAAVSKATNCGLWVAQYANNNHTGYQNKPWNEDAYDCAIRQYSSSGSLPNYGGNLDLNKFYGDAAAWQSYAKSDNQTQQSQEAQQAEPMPAISEITHDGDISTVYVHIPWSVQQDIRMAVVRVGNVVTVNGCGGMSAGDAQWAKANETIPEGFRPTSLSTITLTGGRAALLVQPDGSIYYDGDARDCTTHISGVWITKDTQPK

SEQ ID NO: 24 - protein, 352 aa, Artificial Sequence
  Endolysin polypeptide
MALYIVDVYSGSSDSIIQDPHADGVIVKATQGTSYVNPRCNHQWDLAGQLGKLRGLYHYAGGGNPESEAQYFINNIKNYVGQGILILDWESYQNSSWGDTSWPLRFVTEVHRLTGVWPLIYVQESALWQIANCAPYCGVWVAKYASMDWKSWTLPNMSVSAGAFGALTGWQFTGGDMDRSIFYLTKETWMKIANPSGSKDGWQGSGDTWQYFENGSAVKNDWRKVDNCWYYLDESGNAVTGWKQINNHWYYFDTKHDGSFGTALTGWQQINGKKYYFDLQNAWMLTGKQTIDGKLYTFGDDGALQEATTQETKPAEAKPSIDKQIVLDDQLKAYIQSEIKAQLAKLKIKMEE

SEQ ID NO: 25 - protein, 310 aa, Artificial Sequence
  Endolysin polypeptide
MSLKVVDVYSGSPRWYATDDNADAVIVKATQGTGYVNPFCDIDYQAAKKAGKLLGVYHYASGGDPISEANYFLKNVKGYVHEAILGLDWESAQNASWGNTNWCRQFVNEIHRQTGVWPIIYVQFSAVWQVANCADTCGLWGAGYPWYTNSWTVPPFLSSYNFAPWKDLTGWQFTGNTEDRSLFYVDANGWQAIAKGDGTVTHTAQPAPQKNEAKTSNVPSYAGTTWQDSLGVTWHAENGKFTANTDLHLRWGATPSSSVISVLHAGDVVKYDAWARTNGFVYIRQPRGNGQYGYVAVRNAYTNEAYGKFE

SEQ ID NO: 26 - protein, 305 aa, Artificial Sequence
  Endolysin polypeptide
MALYFTDVFSGSADWIVTDPHAQGTIVKASQGTGYVNPKYEYQYSLAKTNGRLLGLYHYAGGNDPVAEATYFLDHIRGKVGEAVLAVDWEQYQNAAWGNPNWVRKFVDEVHRQTNVWPLIYVQESAIWQVANCANDCGLWVAKYPSMNWHSWQVPDMQVNTAPWPGYTLWQFAGDDEDRSIATVDRNGWQRLANPTGQVSSRPLIFGDSSASSISNNETWTDNLGDTWHREVGRFTSSRPLTLRWGALPTSTAIAELPAGSVIDYDAWSRHDGEVWIRQPRGNGQYGYLPCRNAVTNEAYGTFSE

(19) United States
(12) Patent Application Publication
(10) Pub. No.: US 2024/0066103 A1
(43) Pub. Date: Feb. 29, 2024

(54) POLYPEPTIDES FOR TREATMENT OF BACTERIAL INFECTIONS

(71) Applicant: CC BIOTECH LTD., New Milton Hampshire (GB)

(72) Inventors: Matthew James CUMMINGS, Walton-on-Thames, Surrey (GB); David CORCORAN, London (GB)

(21) Appl. No.: 18/037,632

(22) PCT Filed: Nov. 18, 2020
(86) PCT No.: PCT/EP2020/082604

§ 371 Date: May 18, 2023

(51) Int. Cl. A61K 38/47; C12N 9/36; A61P 31/04

(52) U.S. Cl. CPC A61K 38/47; C12N 9/2462; C12Y 302/01017; A61P 31/04

(57) ABSTRACT

Provided are polypeptides, nucleic acids, vectors, cells and compositions useful for treating bacterial infections, particularly bacterial vaginosis (BV) and related conditions, particularly endolysin polypeptides derived from Gardnerella spp. Also provided is use of amino acid sequences to create a chimeric polypeptide having anti-Gardnerella spp. activity. Methods of treatment using the polypeptides, nucleic acids, vectors, cells and compositions are also provided.

### FIELD

The invention relates to the treatment of bacterial infections, in particular bacterial vaginosis (BV), using novel polypeptides, particularly novel endolysin polypeptides specific for Gardnerella spp. bacteria.

### BACKGROUND

Bacterial vaginosis (BV) affects more than 30% of women of childbearing age (WOCA) in the western world (Koumans, E. H. et al., 2007, Sex. Transm. Dis. 34:864-869). Gardnerella vaginalis is a key pathobiont in BV and is present in over 95% of cases (Schwebke, J. R., et al., 2014, J. Infect. Dis. 210:338-343). Symptomatic BV manifests as a continuous thin-grey discharge and foul fishy odour: these acute symptoms have profound psychological implications for patients. All carriers of G. vaginalis have a ten-fold risk of miscarriage, and two-fold risk of pre-term birth (Leitich, H. et al., 2003, American Journal of Obstetrics and Gynecology 189:139-147). Moreover, BV significantly increases the risk of sexually transmitted infection acquisition (Hay P, 2017, F1000 Res., 6:1761), further negatively impacting female health and fertility.

Metronidazole, clindamycin and secnidazole (Bohbot, J. M., et al., 2010, Infect. Dis. Obstet. Gynecol. 2010:705692) are the front-line therapies currently used to treat BV. However, the condition is poorly managed and recurs frequently, especially during menstruation: 70% of patients experience further symptoms within 9 months of antibiotic treatment (Brotman, R. M. et al., 2007, Journal of Pediatric and Adolescent Gynecology 20:225-231). A thick, sticky bacterial structure known as a biofilm is central to BV recurrence: G. vaginalis is the pioneer and predominant species in polymicrobial BV-associated biofilms. Existing treatments fail to effectively penetrate biofilms. Accordingly, upon cessation of antibiotic treatment, biofilms re-grow, resulting in recurrent symptomatic presentations. Furthermore, existing systemic/vaginal antibiotic regimens reduce the overall vaginal bacterial load, frequently resulting in vaginal candidiasis, and overuse has led to antibiotic resistance in 61% of G. vaginalis clinical isolates (Swidsinski, A. et al., 2008, Am. J. Obstet. Gynecol. 198:97.e1-6; Alves, P., et al. 2014, The Journal of Infectious Diseases 210:593-596; Schuyler, J. A. et al., 2016, Diagn. Microbiol. Infect. Dis. 84:1-3).

Dequalinium chloride (a disinfectant) has been utilised for BV treatment. Pessaries (vaginal suppositories) are inserted for six days, with similar efficacy to antibiotics. However, dequalinium has been associated with opportunistic thrush infection and there is no clinical evidence that it prevents BV recurrence (2017, Drug Ther. Bull. 55:54-57).

Modulation of vaginal pH has been attempted to treat BV, aiming to destroy G. vaginalis and promote growth of healthy vaginal bacteria. Clinical evidence for this approach is poor (Holley, R. L., et al., 2004, Sex. Transm. Dis. 31:236-238; Simoes, J. A. et al., 2006, Br. J. Clin. Pharmacol. 61:211-217). Lactic acid showed best efficacy, but only in combination with metronidazole and no significant reduction in recurrence was observed (Decena, D. C. D. et al., 2006, J. Obstet. Gynaecol. Res. 32:243-251).

Large molecules known as Dendrimers, thought to prevent the attachment of bacteria to the vaginal wall, have recently been developed as therapeutics for treatment of recurrent BV. Evidence for this is poor, with an impractical treatment regime required involving application of the gel every second day for four months (Efficacy and Safety Study of SPL7013 Gel to Prevent the Recurrence of Bacterial Vaginosis (BV)—Full Text View—ClinicalTrials.gov. Available at: https://clinicaltrials.gov/ct2/show/NCT02237950 (Accessed: 17Apr. 2019)) for a 20% reduction in recurrence (Starpharma|Successful VivaGel® Phase 3 results and NDA planned for rBV. Starpharma Available at: https://www.starpharma.com/news/328 (Accessed: 17Apr. 2019)).

Endolysins are polypeptides produced by bacteriophages to digest the host bacterial cell wall and release bacteriophage progeny. Endolysins have been investigated as novel antimicrobial agents to treat bacterial infections in a number of gram-positive species for human health conditions unrelated to bacterial vaginosis. Typically, endolysins are isolated from bacteriophage hosts. Their properties can be modified using common molecular biology and synthetic biology approaches. Native and/or modified endolysins may be administered as therapeutics to disrupt the bacterial cell wall from the outside of the cell and lyse pathogenic bacteria (Love M. J., et al., 2018 Antibiotics (Basel) 7(1):17).

Endolysins normally consist of two domains: a catalytic domain, such as a hydrolase domain (typically located at the N-terminal of the polypeptide), which cleaves specific motifs in the peptidoglycan layer, and a cell wall binding domain (classically located at the C-terminal of the polypeptide), which is involved in specific binding and processing of the bacterial peptidoglycan. Although providing a general organisation for endolysin structure, this typical architecture is not a defined characteristic of all endolysins. Some endolysins can comprise catalytic domains and cell wall binding domains in different orientations. Endolysins that target certain gram negative bacteria may not comprise a dedicated cell wall binding domain. The specificity of an endolysin is often attributed to the cell wall binding domain, which recognizes a cell wall feature specific to the bacteria that it targets.

Bacteriophage endolysins have been investigated as antimicrobial agents to treat bacterial infections and numerous endolysins have been purified and commercialised that target pathogens such as Streptococci spp. and Enterococci spp. in a highly selective fashion (Fenton, M., et al., 2010, Bioengineered Bugs 1:9-16). Endolysins have been shown to be effective in combination with antibiotics, for example. Further, endolysins are able to penetrate and destabilise biofilms where antibiotics are not able to.

Endolysins are typically identified and isolated from characterised bacteriophages. However, no bacteriophages specific for or targeting any members of the Gardnerella Genus, including but not limited to G. vaginalis, G. leopoldii, G. piotii, and G. swidsinskii have been characterised to date.

There remains a need in the art for potent and selective anti-bacterial agents for treating BV that possess a low resistance profile. It would be advantageous for such agents to penetrate and/or destabilise biofilms associated with BV and reduce or prevent recurrence of infection. Preferably, such agents would not affect the balance of flora associated with a healthy vagina.

### SUMMARY

In a first aspect, the invention provides a polypeptide having anti-Gardnerella spp. activity comprising a hydrolase domain.

In a second aspect, the invention provides a polypeptide comprising an amino acid sequence selected from any one of SEQ ID NOs: 1 to 13 or 21 to 26 or a sequence having at least 80, 85, 90, 95, 96, 97, 98, 99, 99.5 or 100% identity thereto or a fragment thereof.

In a third aspect, the invention provides an isolated nucleic acid encoding a polypeptide according to the first or second aspects.

In a fourth aspect, the invention provides a vector comprising an isolated nucleic acid according to the third aspect.

In a fifth aspect, the invention provides a cell comprising a nucleic acid according to the third aspect or vector according to the fourth aspect.

In a sixth aspect, the invention provides a composition comprising a polypeptide according to the first or second aspect, a nucleic acid according to the third aspect, a vector according to the fourth aspect or a cell according to the fifth aspect.

In a seventh aspect, the invention provides the use of at least one amino acid sequence selected from SEQ ID NOs: 1-6 or a sequence having at least 80, 85, 90, 95, 96, 97, 98, 99, 99.5 or 100% identity thereto or a fragment thereof; and/or at least one amino acid sequence selected from SEQ ID NOs: 7-13 or a sequence having at least 80, 85, 90, 95, 96, 97, 98, 99, 99.5 or 100% identity thereto or a fragment thereof to create a chimeric polypeptide having anti-Gardnerella spp. activity.

In a eighth aspect, the invention provides a polypeptide according to the first or second aspect, a nucleic acid according to the third aspect, a vector according to the fourth aspect, a cell according to the fifth aspect or a composition according to the sixth aspect, for use in a method of treating or preventing disease, optionally wherein the disease is a bacterial infection, preferably bacterial vaginosis (BV).

In a ninth aspect, the invention provides a method of treating or preventing a bacterial infection in a subject comprising administering to the subject a therapeutically effective amount of a polypeptide according to the first or second aspect, a nucleic acid according to the third aspect, the vector according to the fourth aspect, a cell according to the fifth aspect or a composition according to the sixth aspect.

### BRIEF DESCRIPTION OF DRAWINGS

FIG. 1 shows a schematic of endolysin structures, showing N-terminal enzymatic active domains (EAD, specifically a hydrolase domain)—and C-terminal cell wall binding domains (CWB). A) full length endolysin comprising 1 EAD (hydrolase) and 2 CWBs. B-C) Endolysin truncations removing one/both CWBs. D-K) Hybrid or chimeric endolysins comprising EAD (hydrolase)/CWB domains and/or mutations and tags to improve bioactivity, substrate selectivity, and solubility. Endolysin polypeptides of the invention can be manipulated into sequences with R1ₐ-Xₙ, R1ₐ-Xₙ—R2_{b}, and R1ₐ-Xₙ—R2_{b}-Yₘ—R3_{c} protein domain architectures, amongst others. R1, R2, R3, Rn, X and Y are as described within this document.

FIG. 2 shows a pictorial description of endolysin curation (step 1)—Example phylogenetic tree built using endolysin candidate CCB2.1. Multiple sequence alignment was built using MEGA6, phylogenetic trees were constructed using maximum likelihood methods and 500 bootstrapped iterations. Bootstrap values>75% are shown. The box represents discrete clades of G. vaginalis endolysin homologues, indicating endolysin selectivity. Furthermore, no endolysin homologues are derived from healthy vaginal commensal bacteria. The dot indicates the last common ancestor between G. vaginalis endolysin sequences and endolysins from some other uropathogens e.g. Atopobium urinale.

FIG. 3 shows a schematic example of endolysin curation (step 2)—Endolysin candidate CCB2.1 was dissected into EAD (hydrolase) and CWB protein domains. A) Homology models for each domain were constructed using SleM (Clostridium perfringen lysozyme, PDB ID:5JIP) and Cpl-7 (Streptococcus Phage CP7 endolysin, PDB ID: 518L) crystal structures. The EAD (hydrolase) and CWB active sites, including peptidoglycan binding residues are shown as balls, all other residues are shown as ribbons. B) Data from A) was further validated using multiple sequence alignments, where conserved active sites and substrate binding residues were manually identified and displayed in shading.

FIG. 4 shows SDS-PAGE and Western blot analysis of the production of the following recombinant proteins of the invention in an appropriate E. coli heterologous host: CCB2.1 (˜36 kDa): a) SDS-PAGE analysis, b) Western blot analysis c) His⁶ tag-free CCB2.1 variant SDS PAGE analysis, CCB2M80 (˜36 kDa): d) SDS-PAGE analysis, e) Western blot analysis, CCB2M81_7 (˜36 kDa): f) SDS-PAGE analysis, g) Western blot analysis, CCB2M83_6 (˜36 kDa): h) SDS-PAGE analysis, l) Western blot analysis, CCB2M84_97 (˜36 kDa): j) SDS-PAGE analysis, k) Western blot analysis, CCB2.3 (˜23 kDa): 1) SDS-PAGE analysis, m) Western blot analysis, n) His⁶tag-free variant SDS-PAGE analysis, CCB3.1: (˜34.6 kDa) o) SDS-PAGE analysis, p) Western blot analysis, CCB6.1 (˜42 kDa): q) SDS-PAGE analysis, r) Western blot analysis, CCB260 (˜44 kDa): s) SDS-PAGE analysis, t) Western blot analysis, CCB230a (˜37 kDa): u) SDS-PAGE analysis, v) Western blot analysis, CCB2M87_2 (˜31 kDa): w) SDS-PAGE analysis, x) Western blot analysis, CCB90_2 (˜36 kDa): y) SDS-PAGE analysis, z) Western blot analysis, CCB2M94_8 (˜36 kDa): aa) SDS-PAGE analysis, ab) Western blot analysis, CCB2.2 (˜30.4 kDa): ac) SDS-PAGE analysis, ad) Western blot analysis, ae) His⁶ tag-free variant SDS PAGE analysis, CCB2.4 (˜25.6 kDa): af) SDS-PAGE analysis, ag) Western blot analysis, CCB3.2 (˜23 kDa): ah) SDS-PAGE analysis, ai) Western blot analysis, CCB4.1 (˜37 kDa): aj) SDS-PAGE analysis, ak) Western blot analysis, CCB4.2 (˜23 kDa): al) SDS-PAGE analysis, am) Western blot analysis, CCB7.1 (˜36 kDa): an) SDS-PAGE analysis, ao) Western blot analysis, CCB8.1 (˜36 kDa): ap) SDS-PAGE analysis, aq) Western blot analysis, CCB280 (˜38 kDa): ar) SDS-PAGE analysis, as) Western blot analysis, CCB270 (˜39 kDa): at) SDS-PAGE analysis, au) Western blot analysis, CCB240 (˜37 kDa): av) SDS-PAGE analysis, aw) Western blot analysis and CCB230b (˜40 kDa): ax) SDS-PAGE analysis, ay) Western blot analysis. Lanes designations are described as the following.

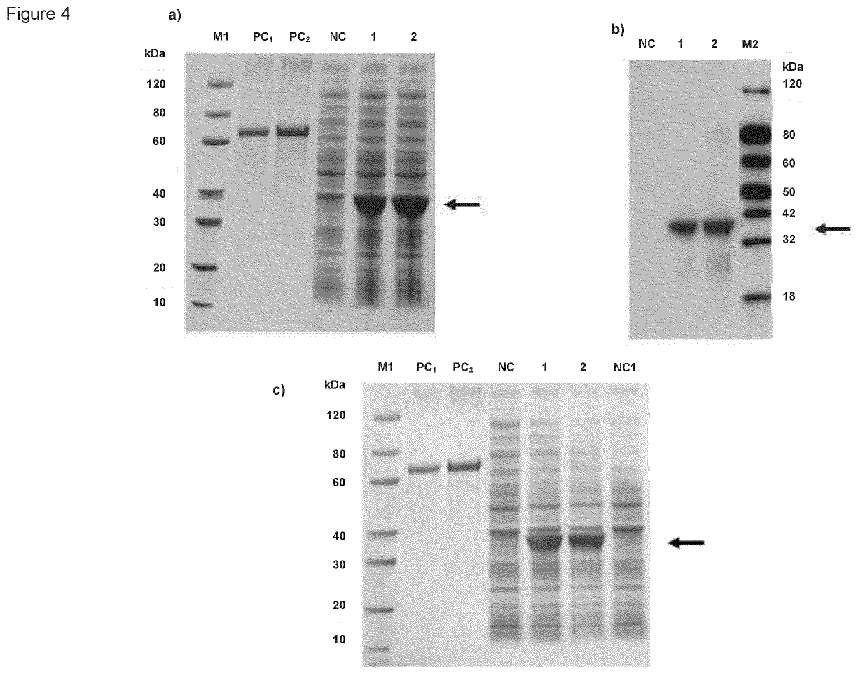

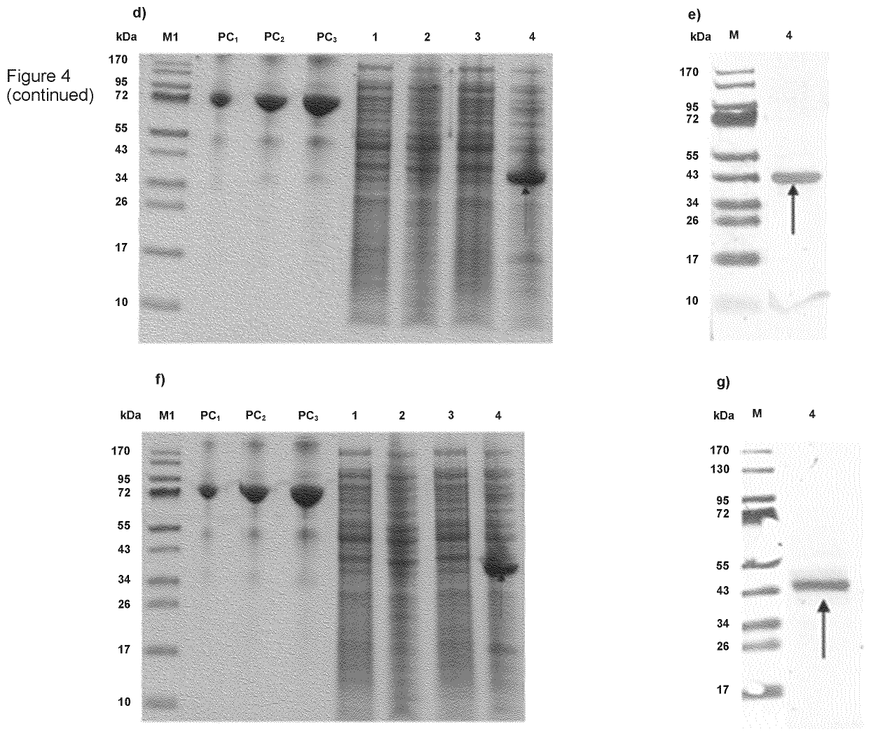

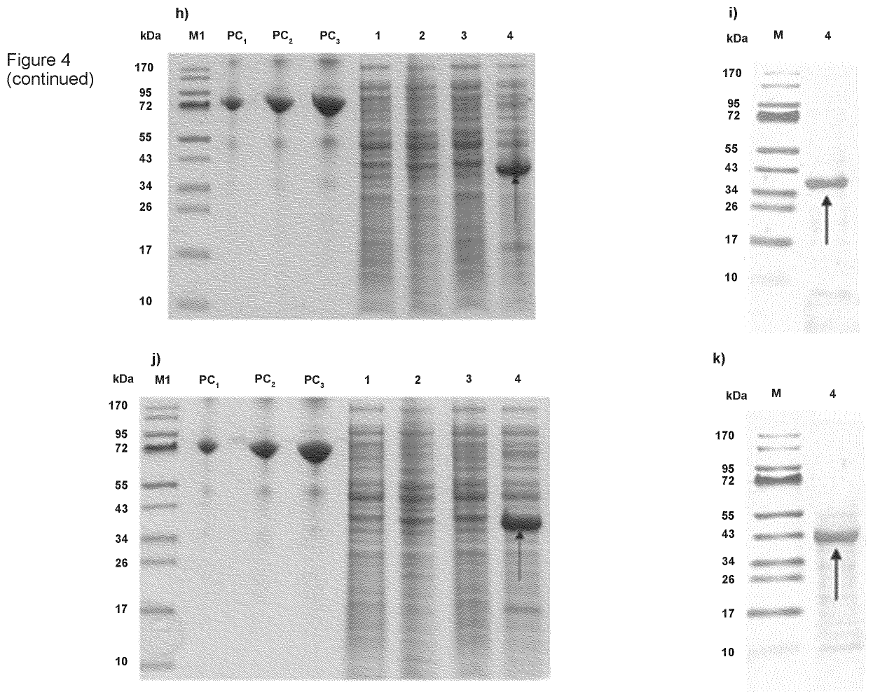

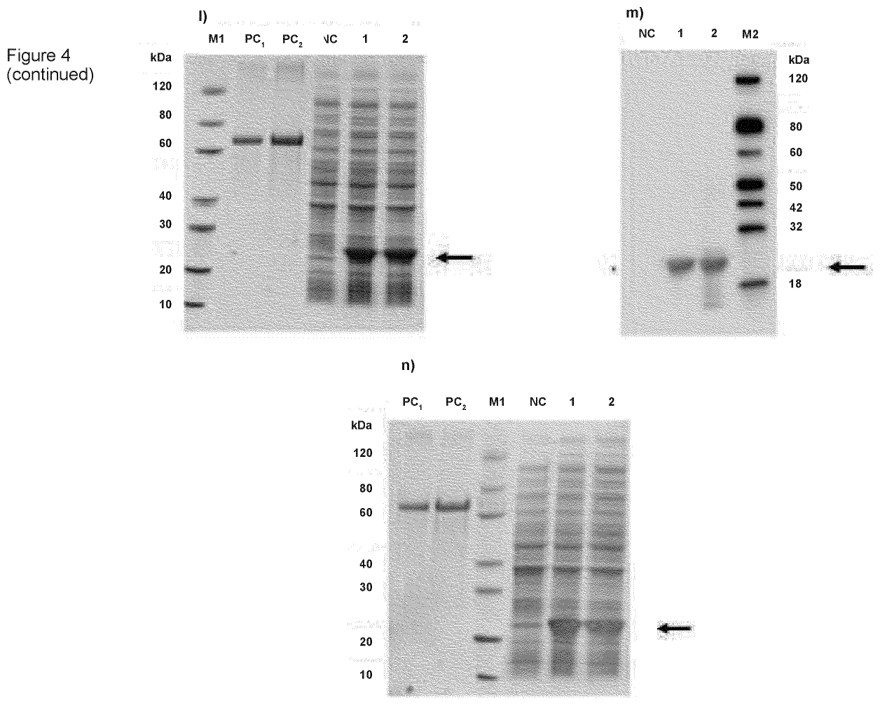

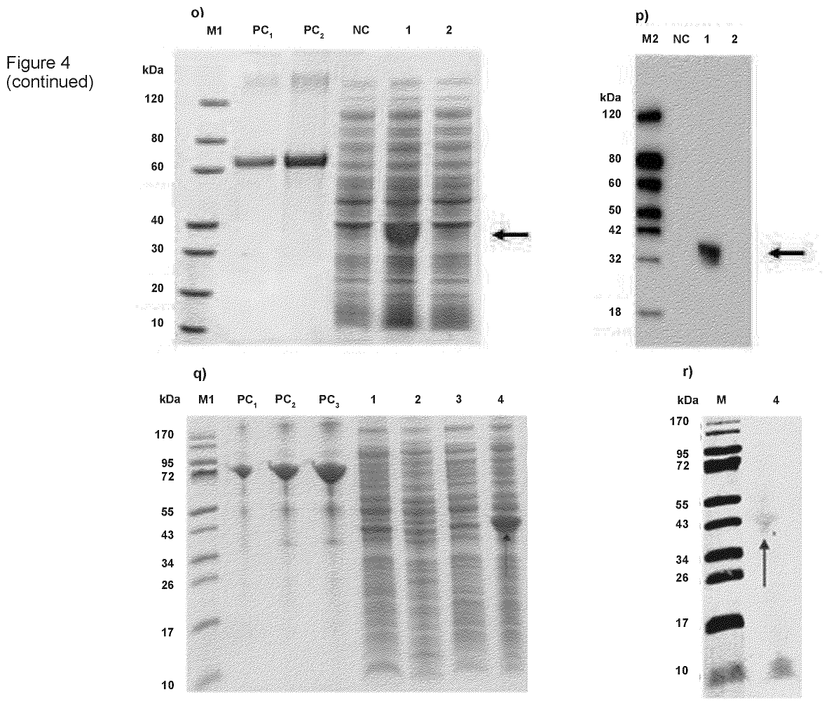

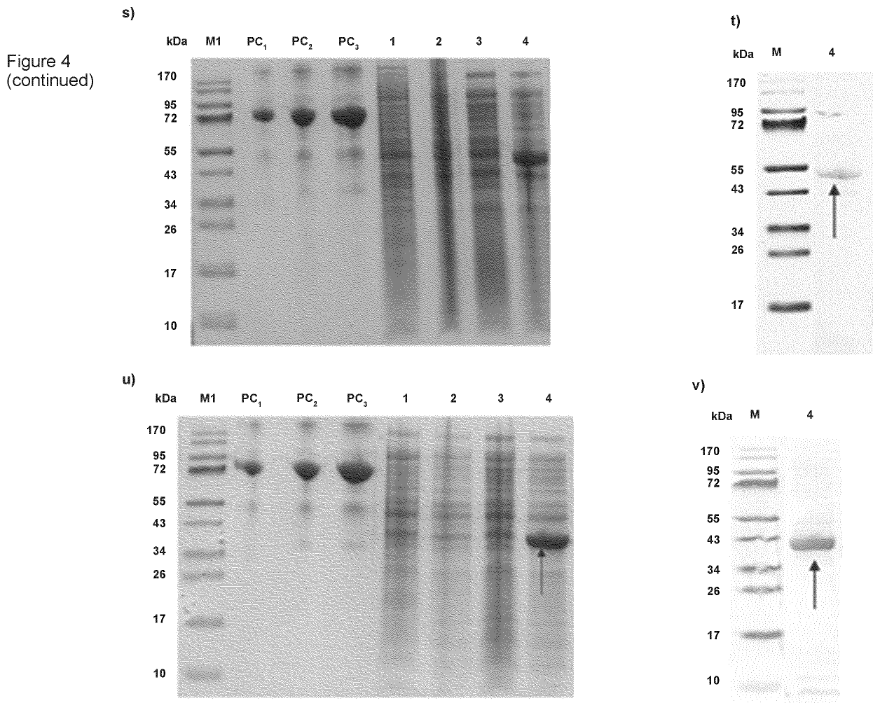

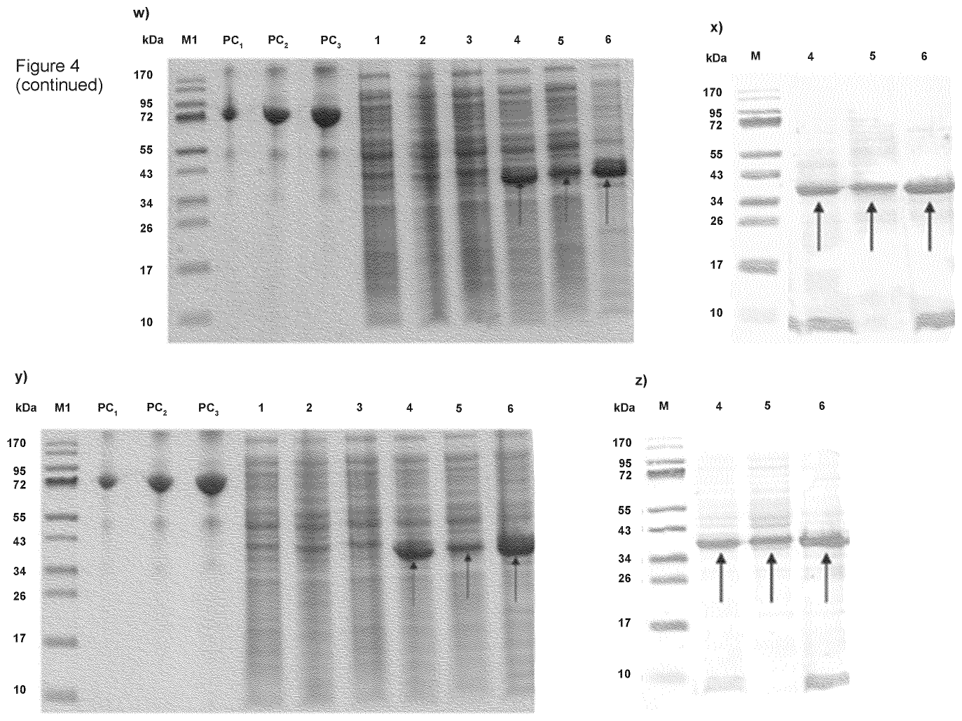

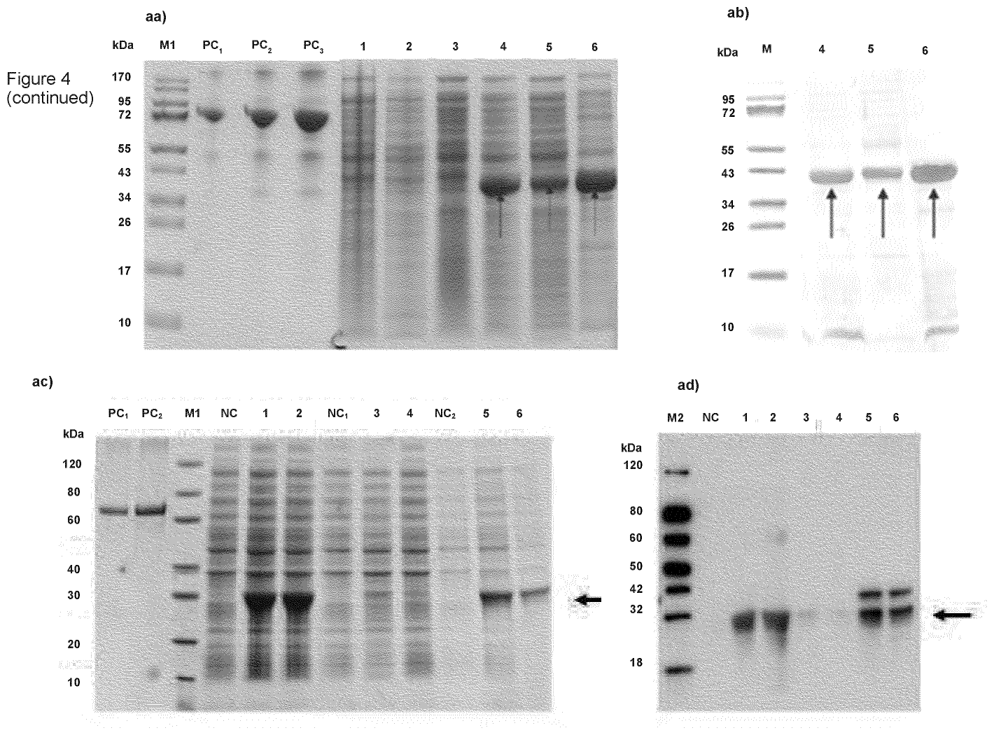

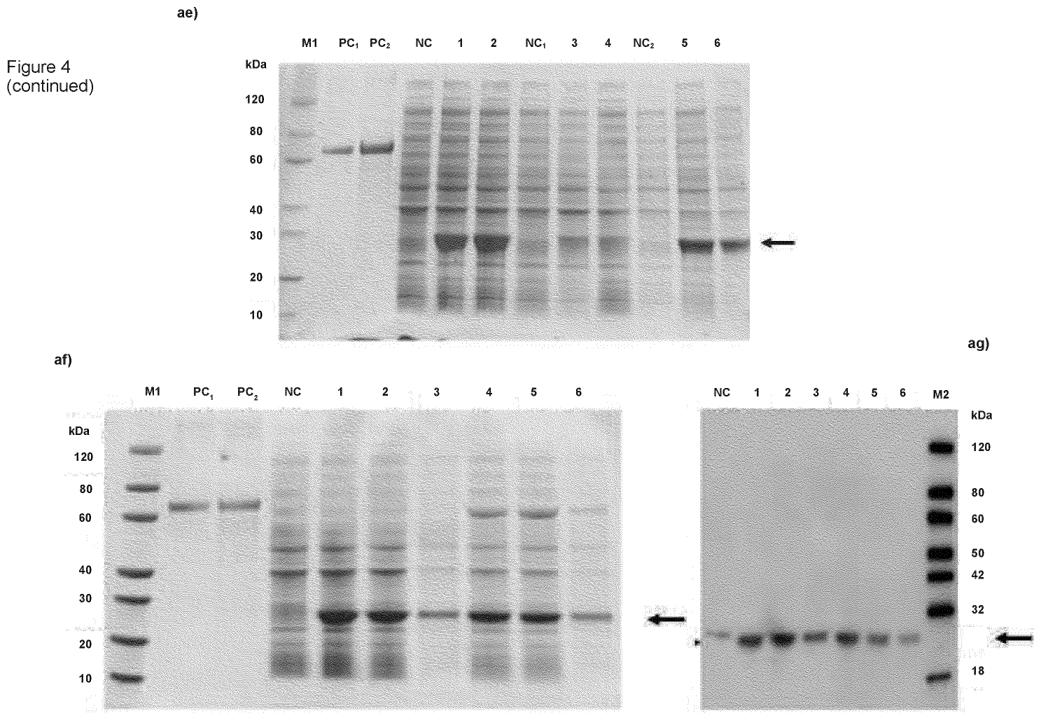

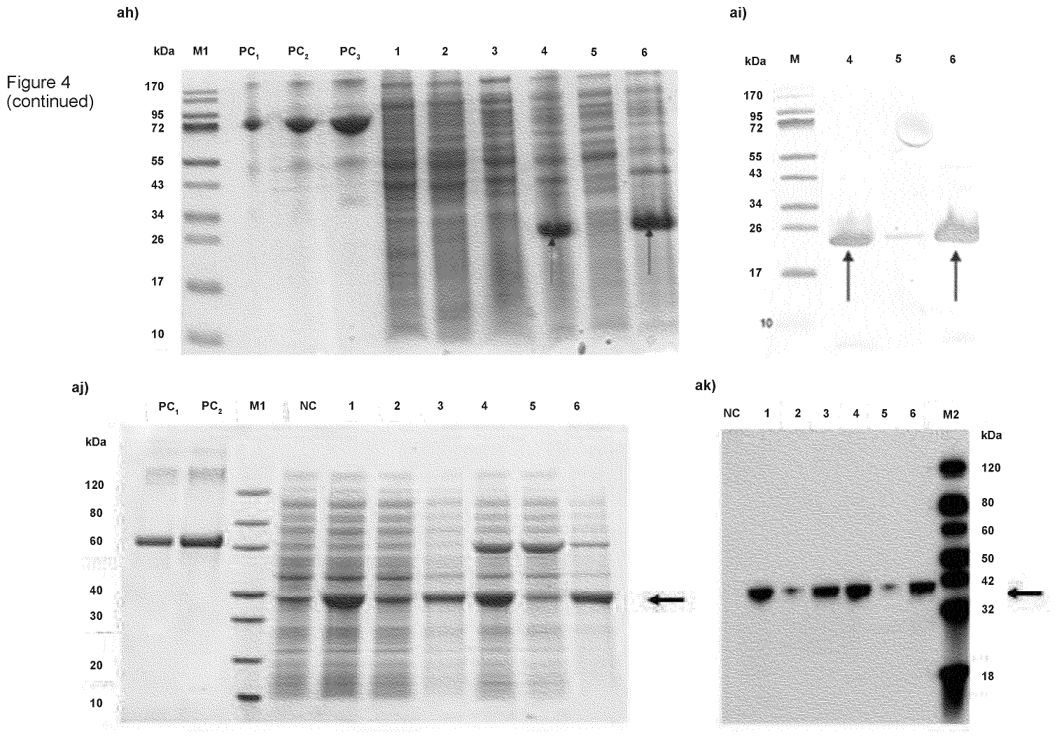

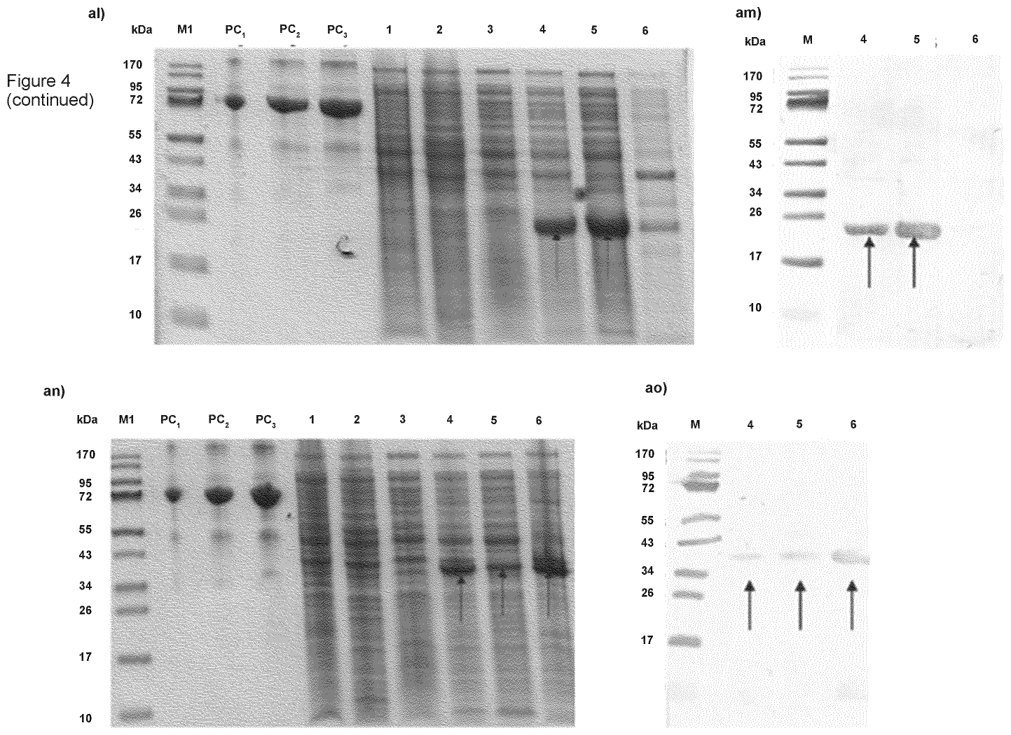

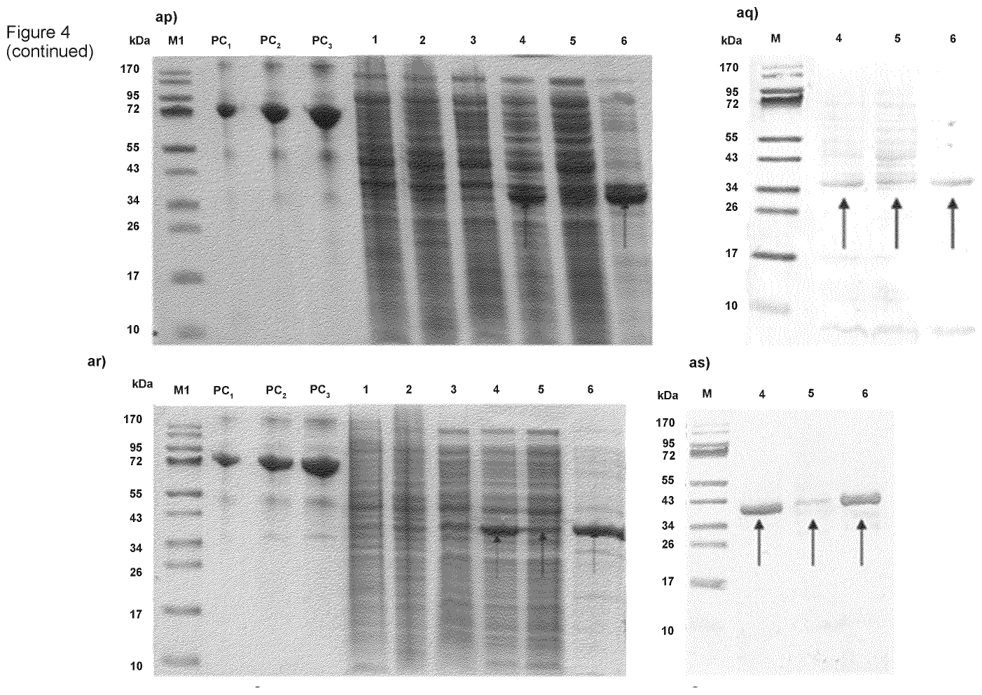

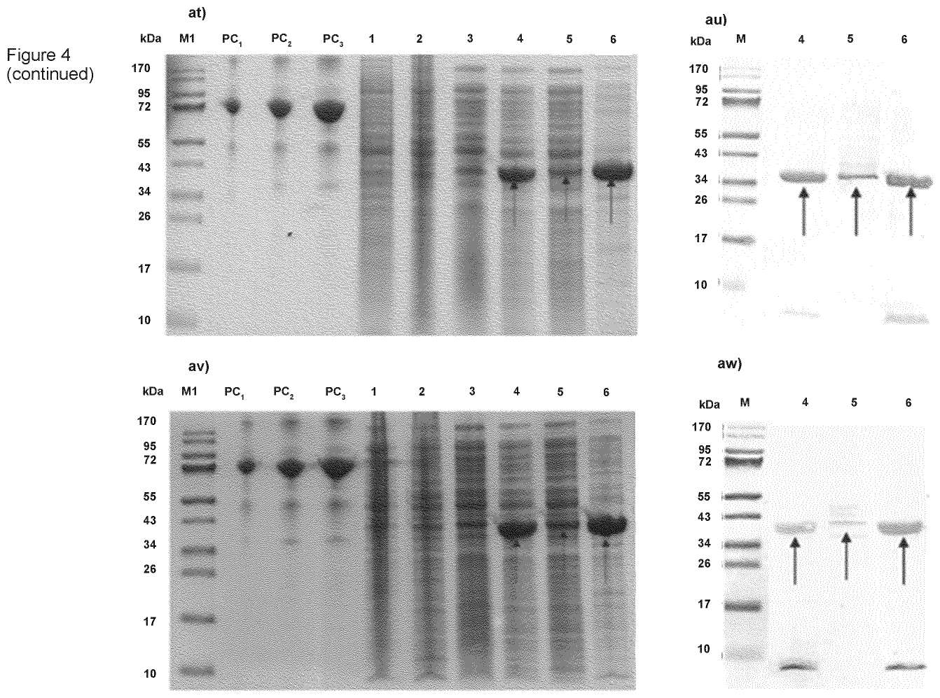

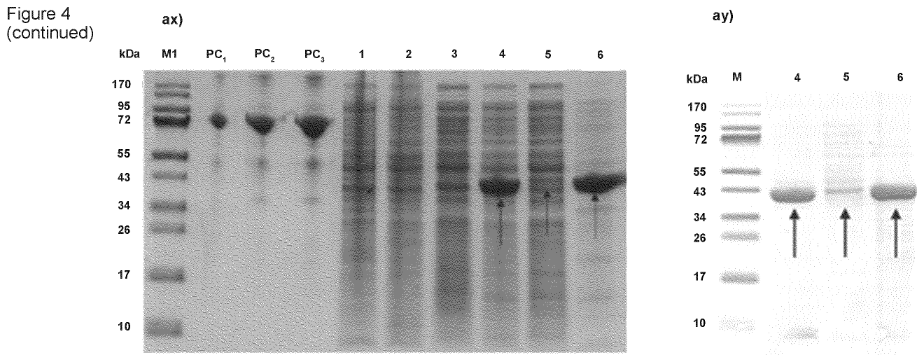

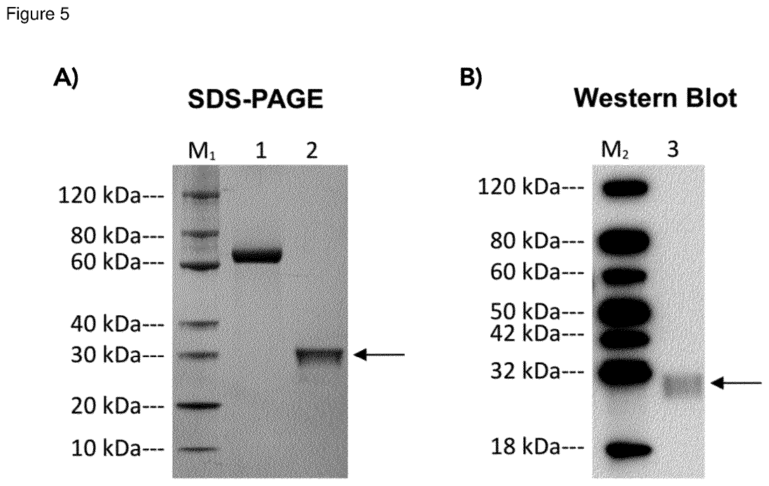

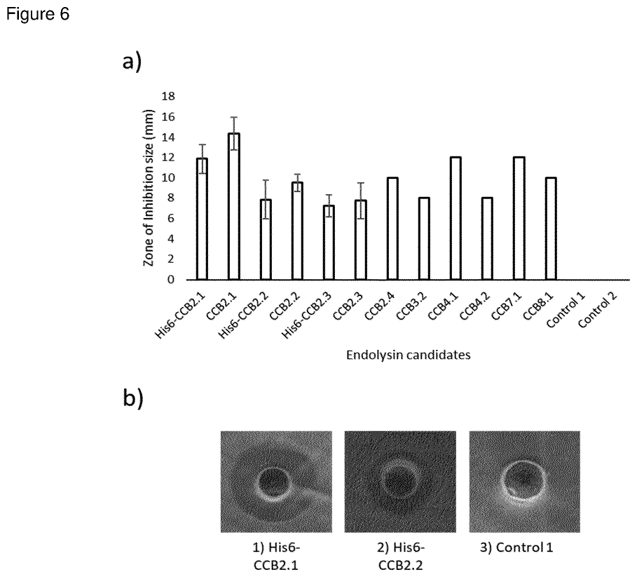

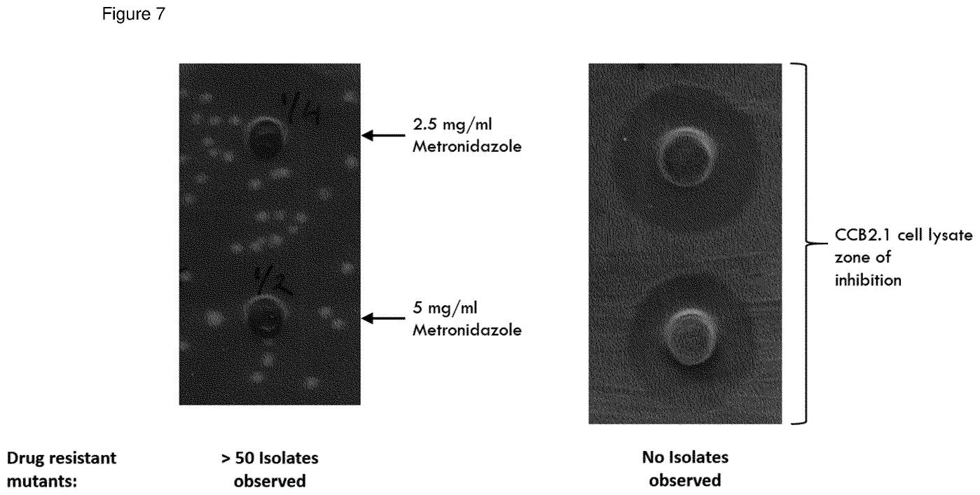

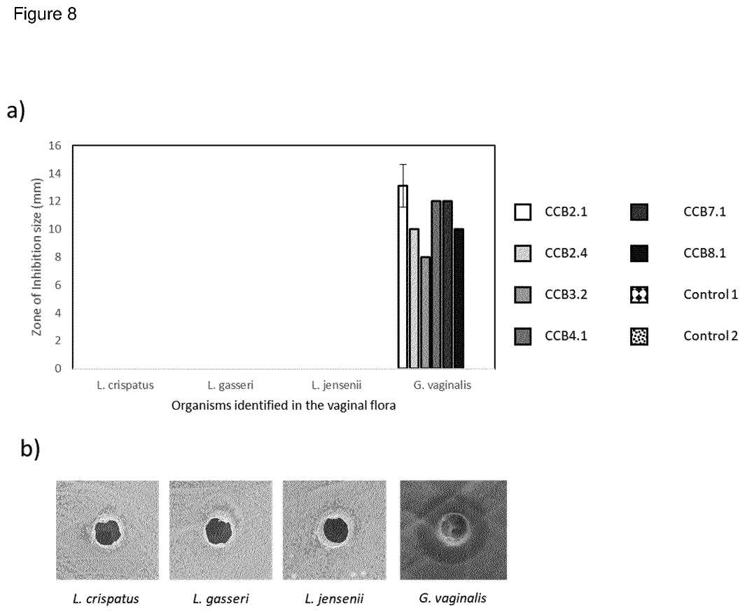

For SDS-PAGE/Western blots labelled in FIG. 4 a-c, l-p: Lane M₁=SDS-PAGE Protein Ladder, molecular weight of protein standards are denoted in kDa, Lane PC₁=Bovine Serum Albumin (BSA) (1 μg), Lane PC₂) BSA (2 μg), Lane NC=Cell lysate of appropriate E. coli heterologous expression host transformed with gene expression vector containing a gene sequence encoding one endolysin polypeptide of the invention without induction, Lane 1=As NC with induction of gene expression and incubation for 16 h at 15° C.), Lane 2=As NC with induction of gene expression for 4 h at 15° C. Lane M₂) Western blot protein ladder, molecular weight of protein standards are denoted in kDa.

SDS-PAGE gels/Western blot analysis labelled ac-ag, aj, and ak share the lane designations of a-c, l-p with additional lane designations of: Lane NC₁=Soluble protein fraction of NC, Lane 3=Soluble protein fraction of Lane 1, Lane 4=Soluble protein fraction of Lane 2, Lane NC₂=Insoluble protein fraction of NC, Lane 5=Insoluble protein fraction of lane 1, Lane 6=Insoluble protein fraction of lane 2.

For SDS-PAGE gels/Western blot analysis labelled in FIG. 4 d-k and q-v: Lane M₁=Protein Ladder, Lane PC₁=Conalbumin (2 μg), Lane PC₂=Conalbumin (5 μg), Lane PC=Conalbumin (10 μg), Lane 1=Cell lysate of E. coli BL21(DE3) or other appropriate expression host without gene expression inducer added to culture medium, Lane 2=Cell lysate of E. coli BL21(DE3) or other appropriate expression host, with induction of gene expression added to culture medium and incubation for 16 h at 16° C., Lane 3=Cell lysate of appropriate E. coli heterologous expression host transformed with gene expression vector containing a gene sequence encoding one endolysin polypeptide of the invention without induction of gene expression, incubated for 16 h at 16° C., Lane 4=lane 3 with induction of gene expression, Lane M₂=Western blot analysis Blot Marker. SDS-PAGE gels/Western blot analysis labelled w-z, aa, ab, ah, ai, al-ay share the lane designations of d-k, q-v with additional lane designations of: Lane 5=Soluble protein fraction of lane 4, Lane 6=Insoluble protein fraction of lane 4. Recombinant protein size is denoted in kDa. Recombinant protein is shown by an arrow. In total, analysis for 24 recombinant endolysin polypeptides of the invention are shown. Western blots were performed using an anti-polyhistidine primary antibody, and appropriate secondary antibody.

FIG. 5 shows SDS-PAGE of CCB2.2 purified from E. coli ArticExpress(DE3) co-expressing cpn10 and cpn60 psychrophilic chaperone genes. CCB2.2 was purified via IMAC from 1 L culture to a final concentration of >16 mg/L at ˜90% purity, as identified by SDS-PAGE and Western blot. Recombinant endolysin protein structures are shown schematically. Purified CCB2.2 contains an N-terminal Hexa-histidine tag, and a TEV protease cleavage site.

FIG. 6 shows anti-Gardnerella bioactivity of twelve endolysin polypeptides of the invention. A) chart displaying CCB2.1, CCB2.2, CCB2.3 and CCB2,4, CCB3.2, CCB4.1, CCB4.2, CCB4.2, CCB7.1 His⁶-CCB2.1, His⁶-CCB2.2 and His⁶-CCB2.3 zone of inhibition on appropriate medium overlaid with Gardnerella vaginalis ATCC 14018. Zone of inhibition are measured in millimeters. Values are an average of multiple studies. Error bars show standard deviation. Zone of inhibition measurements range from 6.5 to 16 mm for experimental samples. All controls show no zone of inhibition. Control 1: normalised cell lysate from an appropriate E. coli heterologous expression host containing an empty expression vector (e.g. E. coli BL21(DE3) pET30a). Control 2: cell lysate sonication buffer (e.g. Buffer 1). Endolysin polypeptides of the invention with a His⁶ prefix comprise an N-terminal Hexa-histidine and TEV protease cleavage site. B) photo of example zone of inhibition.

FIG. 7 shows propensity of Gardnerella vaginalis to develop or not develop resistance to antimicrobial agents, specifically metronidazole and endolysin polypeptides of the invention. Gardnerella vaginalis cultured in the presence of metronidazole (left) at concentrations of 2.5 mg/ml and 5 mg/ml versus E. coli BL21(DE3) cell lysate containing recombinant CCB2.1 (right) for 72 h under anaerobic conditions. Multiple metronidazole resistant G. vaginalis ATCC 14018 colonies are identified. Conversely, no resistance to endolysin polypeptides of the invention was identified in G. vaginalis ATCC 14018.

FIG. 8 shows the selective antimicrobial bioactivity of endolysin polypeptides of the invention towards Gardnerella vaginalis, and not towards common commensal organisms of the vagina, including Lactobacillus gasseri CCUG 31451T, Lactobacillus crispatus CCUG 30722T and Lactobacillus jensenii CCUG 21961T. a) zone of inhibition measurements of normalised cell lysates containing recombinant endolysin polypeptides of the invention, specifically CCB2.1, CCB2.4, CCB3.2, CCB4.1, CCB4.2, CCB7.1 and CCB8.1, normalised cell lysate from an appropriate E. coli heterologous expression host containing an empty expression vector (e.g. E. coli BL21(DE3) pET30a) denoted as control 1, and cell lysate sonication buffer (e.g. Buffer 1), denoted at control 2. Cell lysates containing CCB2.1, CCB2.4 CCB3.2, CCB4.1, CCB4.2, CCB7.1 or CCB8.1 show no antibacterial activity toward any Lactobacillus spp. (Zone of inhibition=0 mm), however, do show anti-Gardnerella spp. bioactivity consistent with previous findings (for example the average zone of inhibition is 13.125 and 9.5 for CCB2.1 and CCB2.4). Controls 1 and 2 show no antimicrobial activity to any bacterial species tested here (zone of inhibition=0 mm). Values are an average of multiple studies. Error bars show standard deviation. b) photo of example zone of inhibition.

### DETAILED DESCRIPTION

The invention described herein is based on the identification, characterisation and generation of novel polypeptides having anti-bacterial activity, specifically activity against Gardnerella spp. bacteria such as G. vaginalis, which makes them useful for treating bacterial infections, such as bacterial vaginosis (BV) or as an adjunct to existing treatments for bacterial infections, such as BV. The polypeptides can comprise or consist of endolysin polypeptides having anti-bacterial activity, preferably those that target Gardnerella spp. bacteria or exhibit activity specifically against Gardnerella spp. bacteria, or fragments thereof that retain such activity.

Traditionally, endolysin discovery involved isolating endolysin gene sequences from characterised lytic bacteriophage particles. However, no bacteriophage particles have been described or characterised that target Gardnerella Spp., specifically G. vaginalis, G. leopoldii, G. piotii, or G. swidsinskii. No endolysin polypeptides have been described that target or have been derived from Gardnerella Spp., specifically G. vaginalis, G. leopoldii, G. piotii, or G. swidsinskii.

Bacteriophage DNA can integrate into host bacterial genomes, only producing viral particles necessary for subsequent infection of bacteria when stressed or challenged. These integrated bacteriophages can become non-viable, i.e., are no longer able to produce infectious viral particles, as a result of host genomic mutations over time.

Utilising a novel approach for identifying therapeutic polypeptides having activity against infectious bacteria, specifically Gardnerella spp. bacteria, the inventors identified the polypeptides of the invention from prophage elements within infectious bacterial genomes, preferably bacteria involved in BV such as Gardnerella spp., preferably G. vaginalis, G. leopoldii, G. piotii, and G. swidsinskii genomes. Prior to the invention, no endolysin polypeptides, in particular endolysins having anti-Gardnerella spp. activity, had been identified and isolated from prophage elements, specifically prophage elements of Gardnerella spp. In one aspect, the invention provides the first endolysin polypeptides targeting Gardnerella spp., specifically targeting G. vaginalis, G. leopoldii, G. piotii, and G. swidsinskii, preferably G. vaginalis.

The invention provides improved treatment options for BV that address the problems identified above, in particular improved treatment options for BV compared to broad-spectrum antibiotics that are the established first-line therapeutic options, for example compared to metronidazole, the standard of care for BV therapy. Specifically, the invention provides polypeptides having improved anti-bacterial activity against bacteria involved in BV such as Gardnerella spp., preferably G. vaginalis, compared to existing therapies. The invention provides polypeptides having improved specificity in targeting bacteria involved in BV, such as Gardnerella spp., preferably G. vaginalis, compared to known therapies. The invention provides polypeptides having lower resistance profiles exhibited by bacteria involved in BV, such as Gardnerella spp., preferably G. vaginalis, compared to known therapies. Notably, the polypeptides of the invention display a reduced or no observable resistance profile. The polypeptides of the invention can penetrate and/or disrupt biofilms comprising or consisting of bacteria involved in BV, such as Gardnerella spp., preferably G. vaginalis and/or reduce or prevent recurrence of infection. Polypeptides of the invention do not affect the balance of flora associated with a healthy vagina, in particular they do not exhibit anti-bacterial activity against healthy vaginal commensal bacteria, for example Lactobacillus spp.

Polypeptides of the invention can comprise a hydrolase domain. Hydrolase enzymes are enzymes that can modify and/or cleave chemical bonds in a substrate. Polypeptides of the invention can comprise a hydrolase domain that can comprise or consist of the full sequence of a hydrolase enzyme or a portion of a hydrolase enzyme that retains hydrolase enzyme activity. Polypeptides of the invention can comprise or consist of a hydrolase domain that can modify and/or cleave a substrate in bacterial cell walls, preferably peptidoglycan. Preferably, the hydrolase domain cleaves peptidoglycan in bacterial cell walls and can cause bacterial cell lysis. Preferably, the hydrolase domain can modify and or cleave bonds that are present in the cell wall and/or peptidoglycan of Gardnerella spp., such as G. vaginalis, G. leopoldii, G. piotii, and/or G. swidsinskii, preferably G. vaginalis. Preferably, the hydrolase domain specifically modifies and/or cleaves a substrate, preferably peptidoglycan, present in the cell wall of Gardnerella spp., such as G. vaginalis, G. leopoldii, G. piotii, and/or G. swidsinskii, preferably G. vaginalis. Suitably, the hydrolase domain does not modify and/or cleave a substrate, preferably peptidoglycan, present in the cell wall of bacteria other than Gardnerella spp., preferably healthy vaginal commensal bacteria, such as Lactobacillus spp. including Lactobacillus crispatus, L. iners, L. jensenii, and L. gasseri. Preferably, polypeptides of the invention can comprise or consist of a hydrolase domain that causes cell lysis of Gardnerella spp. bacteria, such as G. vaginalis, G. leopoldii, G. piotii, and/or G. swidsinskii, preferably G. vaginalis. Suitably, polypeptides of the invention comprise or consist of a hydrolase domain that does not causes cell lysis of bacteria other than Gardnerella spp., preferably healthy vaginal commensal bacteria, such as Lactobacillus spp. including Lactobacillus crispatus, L. iners, L. jensenii, and L. gasseri. Polypeptides of the invention can comprise or consist of a hydrolase domain derived from a bacterial genome, preferably a Gardnerella spp., such as G. vaginalis, G. leopoldii, G. piotii, and/or G. swidsinskii, preferably a G. vaginalis, genome. Polypeptides of the invention can comprise or consist of a hydrolase domain derived from a bacterial endolysin, bacteriophage or prophage, preferably a Gardnerella spp., such as G. vaginalis, G. leopoldii, G. piotii, or G. swidsinskii, preferably G. vaginalis endolysin, bacteriophage or prophage. Polypeptides of the invention can comprise an amidase, glycosidase, hydrolase and/or endo/carboxypeptidase domain, such as a muramidase, glucosaminidase, endopeptidase, and/or N-acetyl-muramoyl-L-alanine amidase domain. Polypeptides of the invention can comprise a glycosyl hydrolase family 25 (GH25; PF01183), Amidase_2 hydrolase (PF01510), glycosyl hydrolase family 73 (GH73; PF01832), Amidase_3 (PF01520), Amidase_5 (PF05382), NLPC_P60 (PF00877), Peptidase_M23 (PF01551), CHAP (PF05257), Lysozyme_like (IPR023346) and/or Transglycoslyase_SLT (PF01464) domain or a plurality or combination thereof, preferably a GH25 domain. These domains can be characterised using typical bioinformatic tools known in the art.

Polypeptides of the invention can comprise or consist of an amino acid sequence as set forth in Table 1, preferably the amino acid sequence of SEQ ID NO: 1, or a sequence having at least 80, 85, 90, 95, 96, 97, 98, 99, 99.5 or 100% amino acid sequence identity thereto, or a fragment thereof.

Polypeptides of the invention can comprise a hydrolase domain as described herein comprising or consisting of an amino acid sequence as set forth in Table 1, preferably the amino acid sequence of SEQ ID NO: 1, or a sequence having at least 80, 85, 90, 95, 96, 97, 98, 99, 99.5 or 100% amino acid sequence identity thereto, or a fragment thereof.

TABLE 1

| | |
|---|---|
| SEQ ID NO: 1 | KKGIDVSEWQGDIDFNAVKASGVEFVIIRAGYGIGCKDKWFEQNYRKAKTCGLDVGAYWY |
| (CCB2.1 | SYANSGFEAAEEAQSCVNMLSGKSFEYPVYFDLEEKSQLNRGRAFCDSLITSFCSKLETY |
| fragment) | GYYAGFYTSLSVANNLVSAHVRNRYALWIAQWNTHCNYQGSYGLWQYSSSGSVPGVAGRV |
| | DMDYAY |
| SEQ ID NO: 2 | QGLKINKIVLHHNAGNLSIQDCYNVWQTREASAHYQVQSDGRIGQLVWDYDTAWHAGNWN |
| (CCB3.1 | ANATSIGIEHADISTSPWQISAQCLENGAHLVAALCKLYGLGRPTWMKNVFPHSYFYATA |
| fragment) | CP |
| SEQ ID NO: 3 | LNGIDISSYQSSINIAAVPADFVIVKATEGTGYINPCFRAHADTILNSGKLLGIYHYISG |
| (CCB4.1 | SGWQAEAEYFVNTVKDYIGRAVLALDFESGGNSAYGDTAYLQQCAQTVYNLTGVHPLIYG |
| fragment) | SQRDYGRLAAVSKATNCGLWVAQYANNNHTGYQNKPWNEDAYDCAIRQYSSSGSLPNYGG |
| | NLDLNKFYGDAAAW |
| SEQ ID NO: 4 | IVDVYSGSSDSIIQDPHADGVIVKATQGTSYVNPRCNHQWDLAGQLGKLRGLYHYAGGGN |
| (CCB6.1 | PESEAQYFINNIKNYVGQGILILDWESYQNSSWGDTSWPLRFVTEVHRLTGVWPLIYVQE |
| fragment) | SALWQIANCAPYCGVWVAKYASMDWKSWTLPNMSVSAGAFGALTGWQFTGGDMDRSIFYL |
| | TKETW |
| SEQ ID NO: 5 | LKVVDVYSGSPRWYATDDNADAVIVKATQGTGYVNPFCDIDYQAAKKAGKLLGVYHYASG |
| (CCB7.1 | GDPISEANYFLKNVKGYVHEAILGLDWESAQNASWGNTNWCRQFVNEIHRQTGVWPIIYV |
| fragment) | QFSAVWQVANCADTCGLWGAGYPWYTNSWTVPPFLSSYNFAPWKDLTGWQFTGNTEDRSL |
| | FYVDANGW |
| SEQ ID NO: 6 | TDVFSGSADWIVTDPHAQGTIVKASQGTGYVNPKYEYQYSLAKTNGRLLGLYHYAGGNDP |
| (CCB8.1 | VAEATYFLDHIRGKVGEAVLAVDWEQYQNAAWGNPNWVRKFVDEVHRQTNVWPLIYVQES |
| fragment) | AIWQVANCANDCGLWVAKYPSMNWHSWQVPDMQVNTAPWPGYTLWQFAGDDEDRSIATVD |
| | RNGW |

Preferably, the polypeptide of the invention can comprise a hydrolase domain as described herein comprising or consisting of an amino acid sequence as set forth in SEQ ID NO: 2, or a sequence having at least 80, 85, 90, 95, 96, 97, 98, 99, 99.5 or 100% amino acid sequence identity thereto, or a fragment thereof.

Preferably, the polypeptide of the invention can comprise a hydrolase domain as described herein comprising or consisting of an amino acid sequence as set forth in SEQ ID NO: 3, or a sequence having at least 80, 85, 90, 95, 96, 97, 98, 99, 99.5 or 100% amino acid sequence identity thereto, or a fragment thereof.

Preferably, the polypeptide of the invention can comprise a hydrolase domain as described herein comprising or consisting of an amino acid sequence as set forth in SEQ ID NO: 4, or a sequence having at least 80, 85, 90, 95, 96, 97, 98, 99, 99.5 or 100% amino acid sequence identity thereto, or a fragment thereof.

Preferably, the polypeptide of the invention can comprise a hydrolase domain as described herein comprising or consisting of an amino acid sequence as set forth in SEQ ID NO: 5, or a sequence having at least 80, 85, 90, 95, 96, 97, 98, 99, 99.5 or 100% amino acid sequence identity thereto, or a fragment thereof.

Preferably, the polypeptide of the invention can comprise a hydrolase domain as described herein comprising or consisting of an amino acid sequence as set forth in SEQ ID NO: 6, or a sequence having at least 80, 85, 90, 95, 96, 97, 98, 99, 99.5 or 100% amino acid sequence identity thereto, or a fragment thereof.

Polypeptides of the invention can comprise a plurality of hydrolase domains as described herein. For example, polypeptides of the invention can comprise one or more hydrolase domains comprising or consisting of an amino acid sequence as set forth in Table 1 or a sequence having at least 80, 85, 90, 95, 96, 97, 98, 99, 99.5 or 100% amino acid sequence identity thereto, or a fragment thereof, optionally further comprising one or more amidase, glycosidase, hydrolase and/or endo/carboxypeptidase domain, such as one or more muramidase, glucosaminidase, endopeptidase, and/or N-acetyl-muramoyl-L-alanine amidase domain, or one or more glycosyl hydrolase family 25 (GH25), Amidase_2 hydrolase, glycosyl hydrolase family 73 (GH73), Amidase_3, Amidase_5, NLPC_P60, Peptidase_M23, CHAP, Lysozyme_like and/or Transglycoslyase_SLT domain.

One or more hydrolase domain can be located at the N-terminal and/or C-terminal region of the polypeptide. Suitably, at least one hydrolase domain can be located at the N-terminal region of the polypeptide. Preferably, one or more hydrolase domains can be located at the N-terminal region of the polypeptide. Polypeptides of the invention can comprise a cell wall binding domain. Cell wall binding domains are polypeptides that interact with and/or bind to bacteria, bacterial cell walls and/or specific substrates within bacterial cell walls, preferably of a given bacterial species or genus. Polypeptides of the invention can comprise a cell wall binding domain that can comprise or consist of the full sequence of a cell wall binding domain or a portion of a cell wall binding domain that retains cell wall binding activity. The cell wall binding domain can comprise any polypeptide capable of specifically binding to bacterial cell walls such as an antibody, antibody fragment (e.g. aptamer) antimicrobial peptide, cell wall anchor protein and/or endolysin cell wall binding domain. Polypeptides of the invention can comprise a CW_7, SH3b, COG5263, SPOR, LysM, PG_Binding_1, PhageMin_tale, Caudovirus_tape_meas_N-terminal, TrusA_like, and/or MFS domain; preferably a CW_7 domain. These domains can be characterised using typical bioinformatic tools known in the art. Polypeptides of the invention can comprise a polypeptide capable of specifically binding to the cell walls of Gardnerella spp., such as G. vaginalis, G. leopoldii, G. piotii, and/or G. swidsinskii, preferably G. vaginalis. Suitably, polypeptides of the invention can comprise a polypeptide capable of specifically binding to the peptidoglycan of Gardnerella spp., such as G. vaginalis, G. leopoldii, G. piotii, and/or G. swidsinskii, preferably G. vaginalis. The cell wall binding domain can comprise a polypeptide derived from a Gardnerella spp., preferably G. vaginalis, genome.

Polypeptides of the invention can comprise or consist of an amino acid sequence as set forth in Table 2, preferably the amino acid sequence of SEQ ID NO: 7 and/or SEQ ID NO: 8, or a sequence having at least 80, 85, 90, 95, 96, 97, 98, 99, 99.5 or 100% amino acid sequence identity thereto, or a fragment thereof. Polypeptides of the invention can comprise a cell wall binding domain as described herein comprising or consisting of an amino acid sequence as set forth in Table 2, preferably the amino acid sequence of SEQ ID NO: 7 and/or SEQ ID NO: 8, or a sequence having at least 80, 85, 90, 95, 96, 97, 98, 99, 99.5 or 100% amino acid sequence identity thereto, or a fragment thereof.

TABLE 2

| | |
|---|---|
| SEQ ID NO: 7 | SSIDEVAREVINGAWGNGKERKQRLTQAGYDYASVQNKVNELL |
| (CCB2.1 | |
| fragment) | |
| SEQ ID NO: 8 | SVDELAREVIRGAWGNGNERKQRLTQAGYDYDTVQKRVNELL |
| (CCB2.1 | |
| fragment) | |
| SEQ ID NO: 9 | SEIVVDGNVSSVEIHVPWGVNNSVGMTLTRAGNVVTANGAGGIKAGDAQWAKANETIP |
| (CCB3.1 | EGFRPTSLSTITLTGGRAALLVQPDGSMYYDGDARDCTTHLSGTWITNDNQPE |
| fragment) | |
| SEQ ID NO: 10 | SEITHDGDISTVYVHIPWSVQQDIRMAVVRVGNVVTVNGCGGMSAGDAQWAKANETIP |
| (CCB4.1 | EGFRPTSLSTITLTGGRAALLVQPDGSIYYDGDARDCTTHISGVWITKDTQPK |
| fragment) | |
| SEQ ID NO: 11 | QFTGGDMDRSIFYLTKETWMKIANPSGSKDGWQGSGDTWQYFENGSAVKNDWRKVDNC |
| (CCB6.1 | WYYLDESGNAVTGWKQINNHWYYFDTKHDGSFGTALTGWQQINGKKYYFDLQNAWMLT |
| fragment) | GKQTIDGKLYTFGDDGAL |
| SEQ ID NO: 12 | ENGKFTANTDLHLRWGATPSSSVISVLHAGDVVKYDAWARTNGFVYIRQPRGNGQYGY |
| (CCB7.1 | VAVR |
| fragment) | |
| SEQ ID NO: 13 | PLTLRWGALPTSTAIAELPAGSVIDYDAWSRHDGEVWIRQPRGNGQYGYLP |
| (CCB8.1 | |
| fragment) | |

Preferably, the polypeptide of the invention can comprise a cell wall binding domain as described herein comprising or consisting of an amino acid sequence as set forth in SEQ ID NO: 9, or a sequence having at least 80, 85, 90, 95, 96, 97, 98, 99, 99.5 or 100% amino acid sequence identity thereto, or a fragment thereof.

Preferably, the polypeptide of the invention can comprise a cell wall binding domain as described herein comprising or consisting of an amino acid sequence as set forth in SEQ ID NO: 10, or a sequence having at least 80, 85, 90, 95, 96, 97, 98, 99, 99.5 or 100% amino acid sequence identity thereto, or a fragment thereof.

Preferably, the polypeptide of the invention can comprise a cell wall binding domain as described herein comprising or consisting of an amino acid sequence as set forth in SEQ ID NO: 11, or a sequence having at least 80, 85, 90, 95, 96, 97, 98, 99, 99.5 or 100% amino acid sequence identity thereto, or a fragment thereof.

Preferably, the polypeptide of the invention can comprise a cell wall binding domain as described herein comprising or consisting of an amino acid sequence as set forth in SEQ ID NO: 12, or a sequence having at least 80, 85, 90, 95, 96, 97, 98, 99, 99.5 or 100% amino acid sequence identity thereto, or a fragment thereof.

Preferably, the polypeptide of the invention can comprise a cell wall binding domain as described herein comprising or consisting of an amino acid sequence as set forth in SEQ ID NO: 13, or a sequence having at least 80, 85, 90, 95, 96, 97, 98, 99, 99.5 or 100% amino acid sequence identity thereto, or a fragment thereof.

Polypeptides of the invention can comprise a plurality of cell wall binding domains as described herein. For example, polypeptides of the invention can comprise one or more cell wall binding domain comprising or consisting of an amino acid sequence as set forth in Table 2 or a sequence having at least 80, 85, 90, 95, 96, 97, 98, 99, 99.5 or 100% amino acid sequence identity thereto, or a fragment thereof, optionally further comprising one or more antibody, antibody fragment (e.g. an aptamer) antimicrobial peptide, cell wall anchor protein and/or endolysin cell wall binding domain, or one or more CW_7, SH3b, COG5263, SPOR, LysM, PG_Binding_1, PhageMin_tale, Caudovirus_tape_meas_N-terminal, TrusA_like, and/or MFS domain.

One or more cell wall binding domain can be located at the C-terminal and/or N-terminal region of the polypeptide. Suitably, at least one cell wall binding domain can be located at the C-terminal region of the polypeptide. Preferably, a plurality of cell wall binding domains can be located at the C-terminal region of the polypeptide. The cell wall binding domain may have catalytic activity, for example the cell wall binding domain may also have hydrolase activity as described herein.

Polypeptides of the invention can comprise one or more linker region. Linkers can include any flexible linker regions known to those skilled in the art (see Briers Y. et al., 2014, mBio 5(4):e01379-14; Chen X. et al., 2013, Adv Drug Deliv Rev. 65(10):1357-69; and Jain N. et al., 2015, Pharm Res. 32(11):3526-40, for example) and can include non-peptide linkers such as poly carbon/poly amide chains or peptide linkers such as those found in endolysins. Preferably, the one or more linker is a peptide sequence, for example, a linker can include from 1 to 100, 1 to 75, 1 to 50, 1 to 25 or 1-10 amino acids; such as about 100, about 50, about 25, about 10, 9, 8, 7, 6, 5, 4, 3, 2 or 1 amino acids.

Polypeptides of the invention can comprise one or more amino acid sequence as set forth in Table 3, preferably the amino acid sequence of SEQ ID NO: 14 and/or SEQ ID NO: 15, or a sequence having at least 80, 85, 90, 95, 96, 97, 98, or 100% amino acid sequence identity thereto, or a fragment thereof. Polypeptides of the invention can comprise one or more linker as described herein comprising or consisting of an amino acid sequence as set forth in Table 3, preferably the amino acid sequence of SEQ ID NO: 14 and/or SEQ ID NO: 15, or a sequence having at least 80, 85, 90, 95, 96, 97, 98, 99, 99.5 or 100% amino acid sequence identity thereto, or a fragment thereof.

TABLE 3

| | |
|---|---|
| SEQ ID NO: 14 | VDYPSIIKNAGLNGCKNGGSDQAART |
| (CCB2.1 | |
| fragment) | |
| SEQ ID NO: 15 | GVKAYRK |
| (CCB2.1 | |
| fragment) | |
| SEQ ID NO: 16 | CSIRDSQNLDYMKRAGDWYDAMTGVVKQQNQTKSGEEYPMIK |
| (CCB3.1 | |
| fragment) | |
| SEQ ID NO: 17 | QSYAKSDNQTQQSQEAQQAEPMPAI |
| (CCB4.1 | |
| fragment) | |
| SEQ ID NO: 18 | QEATTQETKPAEAKPSIDKQIVLDDQLKAYIQSEIKAQLAKLKIKMEE |
| (CCB6.1 | |
| fragment) | |
| SEQ ID NO: 19 | QAIAKGDGTVTHTAQPAPQKNEAKTSNVPSYAGTTWQDSLGVTWHA |
| (CCB7.1 | |
| fragment) | |
| SEQ ID NO: 20 | QRLANPTGQVSSRPLIFGDSSASSISNNETWTDNLGDTWHREVGRFTS |
| (CCB8.1 | SR |
| fragment) | |

Preferably, the polypeptide of the invention can comprise or consist of one or more linker as described herein comprising or consisting of an amino acid sequence as set forth in SEQ ID NO: 16, or a sequence having at least 80, 85, 90, 95, 96, 97, 98, 99, 99.5 or 100% amino acid sequence identity thereto, or a fragment thereof.

Preferably, the polypeptide of the invention can comprise or consist of one or more linker as described herein comprising or consisting of an amino acid sequence as set forth in SEQ ID NO: 17, or a sequence having at least 80, 85, 90, 95, 96, 97, 98, 99, 99.5 or 100% amino acid sequence identity thereto, or a fragment thereof.

Preferably, the polypeptide of the invention can comprise or consist of one or more linker as described herein comprising or consisting of an amino acid sequence as set forth in SEQ ID NO: 18, or a sequence having at least 80, 85, 90, 95, 96, 97, 98, 99, 99.5 or 100% amino acid sequence identity thereto, or a fragment thereof.

Preferably, the polypeptide of the invention can comprise or consist of one or more linker as described herein comprising or consisting of an amino acid sequence as set forth in SEQ ID NO: 19, or a sequence having at least 80, 85, 90, 95, 96, 97, 98, 99, 99.5 or 100% amino acid sequence identity thereto, or a fragment thereof.

Preferably, the polypeptide of the invention can comprise or consist of one or more linker as described herein comprising or consisting of an amino acid sequence as set forth in SEQ ID NO: 20, or a sequence having at least 80, 85, 90, 95, 96, 97, 98, 99, 99.5 or 100% amino acid sequence identity thereto, or a fragment thereof.

Polypeptides of the invention can comprise one or more hydrolase domain, one or more cell wall binding domain or a combination thereof as described herein. Polypeptides of the invention may comprise one or more linker N-terminal and/or C-terminal to the hydrolase domain; one or more linker N-terminal and/or C-terminal to the cell wall binding domain; one or more linker between a plurality of hydrolase domain(s) and/or a plurality of cell wall binding domain(s); preferably a linker C-terminal to the hydrolase domain. Polypeptides of the invention that comprise a plurality of hydrolase domains may comprise one or more linker between each hydrolase domain. Polypeptides of the invention that comprise a plurality of cell wall binding domains may comprise one or more linker between each cell wall binding domain. Alternatively, the one or more hydrolase domain(s) and the one or more cell wall binding domain(s), the plurality of hydrolase domains and/or the plurality of cell wall binding domains may be directly attached (not comprising a linker between the respective domains).

Preferably, the polypeptide of the invention can comprise or consist of at least one amino acid sequence selected from SEQ ID NOs: 1-6 or a sequence having at least 80, 85, 90, 95, 96, 97, 98, 99, 99.5 or 100% identity thereto or a fragment thereof; and/or at least one amino acid sequence selected from SEQ ID NOs: 7-13 or a sequence having at least 80, 85, 90, 95, 96, 97, 98, 99, 99.5 or 100% identity thereto or a fragment thereof to create a chimeric polypeptide having anti-Gardnerella spp. activity

Preferably, the polypeptide of the invention can comprise or consist of at least one amino acid sequence selected from SEQ ID NOs: 1-6 or a sequence having at least 80, 85, 90, 95, 96, 97, 98, 99, 99.5 or 100% identity thereto or a fragment thereof; and/or at least one amino acid sequence selected from SEQ ID NOs: 7-13 or a sequence having at least 80, 85, 90, 95, 96, 97, 98, 99, 99.5 or 100% identity thereto or a fragment thereof and/or at least one amino acid sequence selected from SEQ ID NOs: 14-20 or a sequence having at least 80, 85, 90, 95, 96, 97, 98, 99, 99.5 or 100% identity thereto or a fragment thereof to create a chimeric polypeptide having anti-Gardnerella spp. activity.

Preferably, the polypeptide of the invention can comprise or consist of at least one amino acid sequence selected from SEQ ID NOs: 2-6 or a sequence having at least 80, 85, 90, 95, 96, 97, 98, 99, 99.5 or 100% identity thereto or a fragment thereof; and/or at least one amino acid sequence selected from SEQ ID NOs: 9-13 or a sequence having at least 80, 85, 90, 95, 96, 97, 98, 99, 99.5 or 100% identity thereto or a fragment thereof to create a chimeric polypeptide having anti-Gardnerella spp. activity

Preferably, the polypeptide of the invention can comprise or consist of at least one amino acid sequence selected from SEQ ID NOs: 2-6 or a sequence having at least 80, 85, 90, 95, 96, 97, 98, 99, 99.5 or 100% identity thereto or a fragment thereof; and/or at least one amino acid sequence selected from SEQ ID NOs: 9-13 or a sequence having at least 80, 85, 90, 95, 96, 97, 98, 99, 99.5 or 100% identity thereto or a fragment thereof and/or at least one amino acid sequence selected from SEQ ID NOs: 16-20 or a sequence having at least 80, 85, 90, 95, 96, 97, 98, 99, 99.5 or 100% identity thereto or a fragment thereof to create a chimeric polypeptide having anti-Gardnerella spp. activity.

Preferably, when the hydrolase domain is SEQ ID No: 1 or a sequence having at least 80, 85, 90, 95, 96, 97, 98, 99, 99.5 or 100% identity thereto or a fragment thereof the cell wall binding domain is not SEQ ID No: 7 or 8 or a sequence having at least 80, 85, 90, 95, 96, 97, 98, 99, 99.5 or 100% identity thereto or a fragment thereof.

Preferably, when the cell wall binding domain is SEQ ID No: 7 or 8, or a sequence having at least 80, 85, 90, 95, 96, 97, 98, 99, 99.5 or 100% identity thereto or a fragment thereof the hydrolase domain is not SEQ ID No: 1 or a sequence having at least 80, 85, 90, 95, 96, 97, 98, 99, 99.5 or 100% identity thereto or a fragment thereof.

Preferably, when the hydrolase domain is SEQ ID No: 1 and/or the cell wall binding domain is SEQ ID No: 7 or 8 or a sequence having at least 80, 85, 90, 95, 96, 97, 98, 99, 99.5 or 100% identity thereto the one or more linker is not SEQ ID No: 14 or 15 or a sequence having at least 80, 85, 90, 95, 96, 97, 98, 99, 99.5 or 100% identity thereto or a fragment thereof.

Polypeptides of the invention can comprise or consist of an amino acid sequence as set forth in Table 4, preferably the amino acid sequence of SEQ ID NO: 21, or a sequence having at least 80, 85, 90, 95, 96, 97, 98, 99, 99.5 or 100% amino acid sequence identity thereto, or a fragment thereof.

Polypeptides of the invention can comprise or consist of an endolysin polypeptide as described herein comprising or consisting of an amino acid sequence as set forth in Table 4, preferably the amino acid sequence of SEQ ID NO: 21, 22, 23, 24, 25, 26, 39, 47, 64, 65, 67, 68, 72, 73, 74, 75, 76 or 77, or a sequence having at least 80, 85, 90, 95, 96, 97, 98, 99, 99.5 or 100% amino acid sequence identity thereto, or a fragment thereof. In one embodiment the amino acid sequence is SEQ ID NO: 21, or a sequence having at least 80, 85, 90, 95, 96, 97, 98, 99, 99.5 or 100% amino acid sequence identity thereto, or a fragment thereof. In one embodiment the amino acid sequence is SEQ ID NO: 73, or a sequence having at least 80, 85, 90, 95, 96, 97, 98, 99, 99.5 or 100% amino acid sequence identity thereto, or a fragment thereof. In one embodiment the amino acid sequence is SEQ ID NO: 74, or a sequence having at least 80, 85, 90, 95, 96, 97, 98, 99, 99.5 or 100% amino acid sequence identity thereto, or a fragment thereof. In one embodiment the amino acid sequence is SEQ ID NO: 75, or a sequence having at least 80, 85, 90, 95, 96, 97, 98, 99, 99.5 or 100% amino acid sequence identity thereto, or a fragment thereof.

TABLE 4

| | |
|---|---|
| SEQ ID NO: 21 | MSKKGIDVSEWQGDIDFNAVKASGVEFVIIRAGYGIGCKDKWFEQNYRKAKTCGLDVGAY |
| (CCB2.1) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTSSIDEVAREVINGAWGNGKERKQRLT |
| | QAGYDYASVQNKVNELLGVKAYRKSVDELAREVIRGAWGNGNERKQRLTQAGYDYDTVQK |
| | RVNELL |
| SEQ ID NO: 22 | MKNWETCDADEIKLLTTHYTKGRQGLKINKIVLHHNAGNLSIQDCYNVWQTREASAHYQV |
| (CCB3.1) | QSDGRIGQLVWDYDTAWHAGNWNANATSIGIEHADISTSPWQISAQCLENGAHLVAALCK |
| | LYGLGRPTWMKNVFPHSYFYATACPCSIRDSQNLDYMKRAGDWYDAMTGVVKQQNQTKSG |
| | EEYPMIKSEIVVDGNVSSVEIHVPWGVNNSVGMTLTRAGNVVTANGAGGIKAGDAQWAKA |
| | NETIPEGFRPTSLSTITLTGGRAALLVQPDGSMYYDGDARDCTTHLSGTWITNDNQPE |
| SEQ ID NO: 23 | MALNGIDISSYQSSINIAAVPADFVIVKATEGTGYINPCFRAHADTILNSGKLLGIYHYI |
| (CCB4.1) | SGSGWQAEAEYFVNTVKDYIGRAVLALDFESGGNSAYGDTAYLQQCAQTVYNLTGVHPLI |
| | YGSQRDYGRLAAVSKATNCGLWVAQYANNNHTGYQNKPWNEDAYDCAIRQYSSSGSLPNY |
| | GGNLDLNKFYGDAAAWQSYAKSDNQTQQSQEAQQAEPMPAISEITHDGDISTVYVHIPWS |
| | VQQDIRMAVVRVGNVVTVNGCGGMSAGDAQWAKANETIPEGFRPTSLSTITLTGGRAALL |
| | VQPDGSIYYDGDARDCTTHISGVWITKDTQPK |
| SEQ ID NO: 24 | MALYIVDVYSGSSDSIIQDPHADGVIVKATQGTSYVNPRCNHQWDLAGQLGKLRGLYHYA |
| (CCB6.1) | GGGNPESEAQYFINNIKNYVGQGILILDWESYQNSSWGDTSWPLRFVTEVHRLTGVWPLI |
| | YVQESALWQIANCAPYCGVWVAKYASMDWKSWTLPNMSVSAGAFGALTGWQFTGGDMDRS |
| | IFYLTKETWMKIANPSGSKDGWQGSGDTWQYFENGSAVKNDWRKVDNCWYYLDESGNAVT |
| | GWKQINNHWYYFDTKHDGSFGTALTGWQQINGKKYYFDLQNAWMLTGKQTIDGKLYTFGD |
| | DGALQEATTQETKPAEAKPSIDKQIVLDDQLKAYIQSEIKAQLAKLKIKMEE |
| SEQ ID NO: 25 | MSLKVVDVYSGSPRWYATDDNADAVIVKATQGTGYVNPFCDIDYQAAKKAGKLLGVYHYA |
| (CCB7.1) | SGGDPISEANYFLKNVKGYVHEAILGLDWESAQNASWGNTNWCRQFVNEIHRQTGVWPII |
| | YVQFSAVWQVANCADTCGLWGAGYPWYTNSWTVPPFLSSYNFAPWKDLTGWQFTGNTEDR |
| | SLFYVDANGWQAIAKGDGTVTHTAQPAPQKNEAKTSNVPSYAGTTWQDSLGVTWHAENGK |
| | FTANTDLHLRWGATPSSSVISVLHAGDVVKYDAWARTNGFVYIRQPRGNGQYGYVAVRNA |
| | YTNEAYGKFE |
| SEQ ID NO: 26 | MALYFTDVFSGSADWIVTDPHAQGTIVKASQGTGYVNPKYEYQYSLAKTNGRLLGLYHYA |
| (CCB8.1) | GGNDPVAEATYFLDHIRGKVGEAVLAVDWEQYQNAAWGNPNWVRKFVDEVHRQTNVWPLI |
| | YVQESAIWQVANCANDCGLWVAKYPSMNWHSWQVPDMQVNTAPWPGYTLWQFAGDDEDRS |
| | IATVDRNGWQRLANPTGQVSSRPLIFGDSSASSISNNETWTDNLGDTWHREVGRFTSSRP |
| | LTLRWGALPTSTAIAELPAGSVIDYDAWSRHDGEVWIRQPRGNGQYGYLPCRNAVTNEAY |
| | GTFSE |
| SEQ ID NO: 27 | MSKKGIDVSEWQGDIDFNAVKASGVEFVIIRAGYGIGCKDKWFEQNYRKAKTAGLDVGAY |
| (CCB2.1.1) | WYSYANSASEAAEEAQSCANMLSGKSFEYPVYFDLEEKSQLNRGRAFCDSLITSFCSKLE |
| | TYGYYAGFYTSLSTANNLVSSHVRNRYALWIAQWNTHCSYQGSYGLWQYSSSGSVPGVAG |
| | RVDMDYAYKDYPSIIKNAGLNGCKNGGSDQAARTSSIDEVAREVINGAWGNGNERKQRLT |
| | QAGYDYTSVQNKVNKLLGVKACRKSVDELAREVIRGTWGNGNERKNRLTQAGYDYDTVQK |
| | RVNELL |
| SEQ ID NO: 28 | MSKKGIDVSEWQGDIDFNAVKASGVEFVIIRAGYGIGCKDKWFEQNYRKAKTAGLDVGAY |
| (CCB2.1.2) | WYSYANSSSEAAEEAQSCANMLSGKSFEYPVYFDLEEKSQLNRGRAFCDSLITGFCSKLE |
| | ACGYYAGFYTSLSTANNLVSAHVRNRYALWIAQWNTHCSYQGSYGLWQYSSNGSVPGVAG |
| | RVDMDYAYKDYPSIIKNAGLNGCKNGGSDQAARTSSIDEVAREVINGAWGNGNERKQRLT |
| | QAGYDYTSVQNKVNKLLGVKACRKSVDELAREVIRGTWGNGNERKNRLTQAGYDYDTVQK |
| | RVNELL |
| SEQ ID NO: 29 | MSKKGIDVSEWQGDIDFNAVKASGVEFVIIRAGYGIGCKDKWFEQNYRKAKTAGLDVGAY |
| (CCB2.1.3) | WYSYANSSSEAAEEAQSCVNMLSGKSFEYPVYFDLEEKSQLNRGRAFCDSLITSFCSKLE |
| | TYGYYAGFYTSLSTANNLVSSHVRNRYALWIAQWNTHCSYQGSYGLWQYSSNGSVPGVAG |
| | RVDMDYAYVDYPSIIKNAGLNGYKNGGSYTAPQTSSIDDVAREVINGAWGNGNERKQRLT |
| | QAGYDYTSVQNKVNKLLGVKACRKSVDELAREVIRGTWGNGNERKNRLTQAGYDYDTVQK |
| | RVNELL |
| SEQ ID NO: 30 | MSKKGIDVSEWQGDIDFNAVKASGVEFVIIRAGYGIGCKDKWFEQNYRKAKTCGLDVGAY |
| (CCB2.1.4) | WYSYANSGFEAAEEAQSCVNMLSGKSFEYPVYFDLEEKSQLNRGRAFCDSLITSFCSKLE |
| | TYGYYAGFYTSLSVVNNLVSAHVRDRYALWIAQWNTHCSYQGSYGLWQYSSSGSVPGVAG |
| | RVDMDYAYKDYPSIIKNAGLNGCKNGGSHQAARTSSIDEVAREVINGAWGNGNERKQRLT |
| | SAGYDYASVQNKVNKLLGVKACRKSVDELAREVIRGTWGNGSTRKQRLTSAGYDYDTVQK |
| | RVNELL |
| SEQ ID NO: 31 | MSKKGIDVSEWQGDIDFNAVKASGVEFVIIRAGYGIGCKDKWFEQNYRKAKTCGLDVGAY |
| (CCB2.1.5) | WYSYANSGFEAAEEAQSCVNMLSGKSFEYPVYFDLEEKSQLNRGRAFCDSLITSFCSKLE |
| | TYGYYAGFYTSLSVVNNLVSAHVRDRYALWIAQWNTHCSYQGSYGLWQYSSSGSVPGVAG |
| | RVDMDYAYVDYPSIIKNVGLNGCKNGGSDQATRTSSIDEVAREVINGAWGNGNERKQRLT |
| | SAGYDYASVQNKVNKLLGVKAYRKSVDELAREVIRGTWGNGNERKQRLAQAGYDYDTVQK |
| | RVNELL |
| SEQ ID NO: 32 | MSKKGIDVSEWQGDIDFNAVKASGVEFVIIRAGYGIGCKDKWFEQNYRKAKTAGLDVGAY |
| (CCB2.1.6) | WYSYANSGSEAAEEAQSCVNTLSGKSFEYPVYFDLEEKSQLNRGRAFCDSLITSFCSKLE |
| | TYGYYAGFYTSLSVANNLVSSHVRNRYALWIAQWNTHCSYQGSYGLWQYSSNGSVPGVAG |
| | RVDMDYAYVDYPSIIKNVGLNGCKNGGSDQAARTSSIDEVVREVINGAWGNGNERKQRLT |
| | SAGYDYASVQNKVNELLGVKACRKSVDELAREVIRGAWGNGSTRKQRLTSAGYDYATVQK |
| | RVNELL |
| SEQ ID NO: 33 | MSKKGIDVSEWQGDIDFNAVKASGIEFVIIRAGYGIGCKDKWFEQNYRKAKTAGLDVGAY |
| (CCB2.1.7) | WYSYANSAGEASEEAQSCVNMLSGKSFEYPVYFDLEEKSQLNRGRVFCDSLITSFCSKLE |
| | ACGYYAGFYTSLSVANNLVSSHVRNRYALWIAQWNTHCSYQGSYGLWQYSSSGSVPGVAG |
| | RVDMDYAYVDYPSVIKNAGLNGCKNGGSDQATRTSSIDEVAREVINGAWGNGNERKQRLT |
| | SAGYDYASVQNKVNKLLGVKTRRKSVDELAREVIRGTWGNGNERKQRLTQAGYDYATVQK |
| | RVNELL |
| SEQ ID NO: 34 | MSKRGIDVSEWQGDIDFNAVKASGVEFVIIRAGYGIGCKDKWFEQNYRKAKTCGLDVGAY |
| (CCB2.1.8) | WYSYANSGFEAAEEAQSCVNMLSGKSFEYPVYFDLEEKSQLNRGRAFCDSLITGFCNKLE |
| | SCGYYAGFYTSLSTANNLVSAHVRNRYALWIAQWNTHCSYQGSYGLWQYSSSGSVPGVAG |
| | RVDMDYAYVDYPSIIKNVGLNGCKNGGSDQAARTSSIDEVAREVINGAWGNGNERKQRLT |
| | SAGYDYASVQNKVNELLGVKACRKSVDEIAREVIRGTWGNGSTRKQRLTQAGYDYDTVQK |
| | RVNELL |
| SEQ ID NO: 35 | MSKKGIDVSEWQGDIDFNAVKASGIEFVIIRAGYGIGCKDKWFEQNYRKAKTVGLDVGAY |
| (CCB2.1.9) | WYSYANSSSEAAEEAQSLMNMLSGKSFEYPVYFDLEEKSQLNRGRAFCDSLITSFCNKLE |
| | DCGYYAGFYTSLSTANNLVSAHVRNRYALWIAQWNTHCSYQGSYGLWQYSSSGSVPGVAG |
| | RVDMDYAYVDYPSIIKNAGLNGCKNGGSDQAARTSSIDEVAREVINGAWGNGSTRKQRLT |
| | SAGYDYASVQNKVNELLGVKACRKSVDELAREVIRGAWGNGSTRKQRLAQAGYDYDTVQK |
| | RVNELL |
| SEQ ID NO: 36 | MSKKGIDVSEWQGDIDFNAVKASGVEFVIIRAGYGIGCKDKWFEQNYRKAKTAGLDVGAY |
| (CCB2.1.10) | WYSYANSGFEAAEEAQSLMNMLSGKSFEYPVYFDLEEKSQLNRGRAFCDSLITSFCNKLE |
| | ACGYYAGFYTSLSTANNLVSAHVRNRYALWIAQWNTHCNYQGSYGLWQYSSNGSVPGVAG |
| | RVDMDYAYVDYPSIIKNAGLNGCKNGGSDQAARTSSIDEVAREVINGAWGNGSTRKQRLT |
| | SAGYDYASVQNKVNELLGVKACRKSVDELAREVIRGAWGNGSTRKQRLAQAGYDYDTVQK |
| | RVNELL |
| SEQ ID NO: 37 | MSKKGIDVSVWQGDIDFNAVKASGVEFVIIRAGYGIECKDKWFEQNYRKAKTAGLDVGAY |
| (CCB2.1.11) | WYSYANSGFEAAEEAQSCVNMLSGKSFEYPVYFDLEEKSQLNRGRAFCDSLITSFCNKLE |
| | SCGYYAGFYTSLSTANNLVPAHVRNRYALWIAQWNTHCDYQGSYGLWQYSSSGSVPGVAG |
| | RVDMDYAYVDYPSIIKNAGLNGYKNGESHQATRTTSIDEVAREVINGAWGNGNERKQRLT |
| | QAGYDYASVQNKVNELLGVKACRKSVDELAREVIRGTWGNGNERKNRLTSAGYDYDTVQK |
| | RVNELL |
| SEQ ID NO: 38 | MSKKGIDVSVWQGDIDFNAVKASGVEFVIIRAGYGIGCKDKWFEQNYRKAKTCGLDVGAY |
| (CCB2.1.12) | WYSYANSGFEAAEEAQSCVNMLSGKSFEYPVYFDLEEKSQLNRGRAFCDSLITSFCNKLE |
| | ACGYYAGFYTSLSTANNLVSAHVRNRYALWIAQWNTHCSYQGSYGLWQYSSSGSVPGVAG |
| | RVDMDYAYVDYPSIIKNAGLNGYKNGESHQATRTTSIDEVAREVINGAWGNGNERKQRLT |
| | SAGYDYASVQNKVNELLGVKACRKSVDELAREVIRGAWGNGSTRKQRLTSAGYDYDTVQK |
| | RVNELL |
| SEQ ID NO: 39 | MSKRGIDVSVWQGDIDFNAVKASGVEFVIIRAGYGIGHKDKWFEENYRKAKTAGLDVGAY |
| (CCB2M90_2) | WYSYASSAGEASEEAQSCVNILSGKSFEYPIYFDLEEKSQLNRGRAFCDSLITSFCSKLE |
| | TYGYYAGFYTSLSVANNLVSSHVRDRYALWIAQWNTHCDYQGSYGLWQYSSSGSVDGIAG |
| | RVDMDYAYVDYPNVIKNAGLNGYKNGGSYTAPQTSSIDEVAREVINGDWGNGNERKNRLT |
| | QAGYDYTSVQNKVNELLGVKAYRKSVDELAREVIRGTWGNGSTRKQRLTQAGYDYDTVQK |
| | RVNELL |
| SEQ ID NO: 40 | MSKKGIDVSVWQGDIDFNSVKASGVEFVIIRAGYGIGHKDKWFEENYRKAKTAGLDVGAY |
| (CCB2.1.13) | WYSYASSAGEASEEAQSCVNILSGKSFEYPVYFDLEEKSQLNRGRAFCDSLITSFCNKLE |
| | TYGYYAGFYTSLSVANNLVSSHVRDRYALWIAQWNTHCDYQGSYGLWQYSSSGSVGGIAG |
| | RVDMDYAYVDYPSVIKKAGLNGYKNGGSYTAPQTSSIDEVAREVINGDWGNGTERKNRLT |
| | SAGYDYTSVQNKVNELLGVKAYRKSVDELAREVIRGTWGNGSMRKQRLTQAGYDYDAVQK |
| | RVNELL |
| SEQ ID NO: 41 | MSKRGIDVSVWQGDIDFNAVKASGVEFVIIRAGYGIGHKDKWFEQNYRKAKTTGLDVGAY |
| (CCB2.1.14) | WYSYASSAGEAAEEAQSCVNILSGKSFEYPVYFDLEEKSQLNRGRDFCDSLITSFCNKLE |
| | TYGYYAGFYTSLSVANNLVSSHVRDRYALWIAQWNTHCDYQGSYGLWQYSSSGSVDGIAG |
| | RVDMDYTYVDYPSVIKKAGLNGYKNGGSYTAPQTSSIDEVAREVINGDWGNGNERKNRLT |
| | SAGYDYTSVQNKVNELLGVKAYRKSVDELAREVIRGTWGNGSTRKQRLTQAGYDYDAVQK |
| | RVNELL |
| SEQ ID NO: 42 | MSKKGIDVSVWQGDIDFNAVKASGVEFVIIRAGYGIGHKDKWFEENYRKAKTAGLDVGSY |
| (CCB2.1.15) | WYSYASSAGEASLEAQSCANILSGKSFEYPIYFDLEEKSQLNRGRAFCDSLITSFCNKLE |
| | TCGYYAGFYTSLSVANNLVSAHVRNRYALWIAQWNTHCDYQGSYGLWQYSSSGSVGGIAG |
| | RVDMDYAYVDYPSVIKNAGLNGYKNGGSYTAPQTSSIDEVAREVINGDWGNGNERKNRLT |
| | SAGYDYASVQNKVNELLGVKAYRKSVDELAREVIRGTWGNGSTRKQRLTQAGYDYDAVQK |
| | RVNELL |
| SEQ ID NO: 43 | MSKKGIDVSVWQGDIDFNAVKASGVEFVIIRAGYGIGCKDKWFEENYRKAKTAGLDVGAY |
| (CCB2.1.16) | WYSYASSAGEADLEAQSCVNILSGKSFEYPVYFDLEEKSQLNRGRDFCDSLITSFCNKLE |
| | ACGYYAGFYTSLSVANNLVSSHVRDRYALWIAQWNTHCSYQGSYGLWQYSSSGSVNGIAG |
| | RVDMDYAYVDYPSVIKNAGLNGYQNGGSYTAPQTSSIDEVAREVINGDWGNGNERKNRLT |
| | SAGYDYTSVQNKVNELLGVKAYRKSVDELAREVIRGTWGNGNTRKQRLTQAGYDYNAVQK |
| | RVNELL |
| SEQ ID NO: 44 | MSKRGIDVSVWQGDIDFNAVKASGVEFVIIRAGYGIGHKDKWFEENYRKAKTAGLDVGAY |
| (CCB2.1.17) | WYSYASSAGEASEEAQSCVNILSGKSFEYPVYFDLEEKSQLNRGRAFCDSLITSFCNKLE |
| | ACGYYAGFYTSLSVANNFVSAHIRDRYALWIAQWNTHCSYQGSYGLWQYSSSGSVNGIAG |
| | RVDMDYAYVDYPSVIKTAGLNGYQNGGSHTAPQTSSIDEVAREVINGDWGNGNERKNRLT |
| | SAGYDYTSVQNKVNELLGVKAYRKSVDELAREVIRGTWGNGNTRKQRLTQAGYDYNAVQK |
| | RVNELL |
| SEQ ID NO: 45 | MSKRGIDVSVWQGDIDFNAVKASGVEFVIIRAGYGIGHKDKWFEENYRKAKTVGLDVGAY |
| (CCB2.1.18) | WYSYASSAGEASEEAQSCVNILSGKSFEYPVYFDLEEKSQLNRGRDFCDSLITSFCNKLE |
| | ACGYYAGFYTSLSVANNLVSSHVRDRYALWIAQWNTHCSYQGSYGLWQYSSSGSVNGIAG |
| | RVDMDYAYVDYPSVIKNAGLNGYKNGGSYTAPQISSIDEVAREVINGDWGNGNERKQRLT |
| | SAGYDYASVQNKVNELLGVKAYRKSVDELAREVIRGTWGNGSTRKQRLTQAGYDYNAVQK |
| | RVNELL |
| SEQ ID NO: 46 | MSKKGIDVSVWQGDIDFNAVKASGVEFVIIRAGYGIGHKDKWFEENYRKAKTAGLDVGSY |
| (CCB2.1.19) | WYSYASSAGEAALEAQSCVNILSGKSFEYPVYFDLEEKSQLNRGRDFCDSLITSFCNKLE |
| | ACGYYAGFYTSLSVANNLVSSHVRDRYALWIAQWNTHCSYEGSYGLWQYSSSGSVNGIAG |
| | RVDMDYAYVDYPSVIKNAGLNGYQNGGSYTAPQTSSIDEVAREVINGDWGNGNERKNRLT |
| | STGYDYTSVQNKVNELLGVKAYRKSVDELAREVIRGTWGNGSTRKQRLTQAGYDYDAVQK |
| | RVNELL |
| SEQ ID NO: 47 | MSKKGIDVSVWQGDIDFNSVKASGVEFVIIRAGYGIGHKDKWFEENYRKAKTAGLDVGSY |
| (CCB2M87_2) | WYSYASSAGEASEEAQSCVNILSGKSFEYPIYFDLEEKSQLNRGRDFCDSLITSFCNKLE |
| | ACGYYAGFYTSLSVANNLVSSHVRDRYALWIAQWNTHCSYQGSYGLWQYSSSGSVNGIAG |
| | RVDMDYAYVDYPSVIKNAGLNGYQNGGSYTAPQTSSIDEVAREVINGDWGNGNDRKNRLI |
| | SAGYDYASVQNKVNELLGVKAYRKSVDELAREVIRGTWGNGSMRKHRLTQAGYDYDAVQK |
| | RVNELL |
| SEQ ID NO: 48 | MSKKGIDVSVWQGDIDFNAVKASGVEFVIIRAGYGIGHKDKWFEENYRKAKTAGLDVGSY |
| (CCB2.1.20) | WYSYASSAGEVALEAQSCVNILSGKSFEYPVYFDLEEKSQLNRGRDFCDSLITSFCNKLE |
| | ACGYYAGFYTSLSVANNLVSSHVRDRYALWIAQWNTHCSYQGSYGLWQYSSSGSVNGIAG |
| | RVDMDYAYVDYPSVIKNAGLNGYQNGGSYTAPQTSSIDEVAREVINGDWGNGIERKNRLT |
| | SAGYDYTSVQNKVNELLGVKAYRKSVDELAREVIRGTWGNGKTRKQRLTQAGYDYNAVQK |
| | RVNELL |
| SEQ ID NO: 49 | MSKKGIDVSEWQGDIDFNAVKASGVEFVIIRAGYGIGCKDKWFEQNYRKAKTAGLDVGAY |
| (CCB2.1.21) | WYSYANSGFEAAEEAQSCANMLSGKSFEYPVYFDLEEKSQLNRGRAFCDSLITSFCNKLE |
| | TCGYYAGFYTSLSTANNLVSSHVRNRYALWIAQWNTHCSYQGSYGLWQYSSSGSVPGVAG |
| | RVDMDYAYVDYPSIIKNAGLNGCKNGGSDQAARTSSIDEVAREVINGAWGNGSTRKQRLT |
| | SAGYDYATVQKRVNELL |
| SEQ ID NO: 50 | MSKKGIDVSEWQGDIDFNAVKASGVEFVIIRAGYGIGCKDKWFEQNYRKAKTCGLDVGAY |
| (CCB2.1.22) | WYSYANSGFEAAEEAQSCVNMLSGKSFEYPVYFDLEEKSQLNRGRVFCDSLITSFCNKLE |
| | ACGYYAGFYTSLSTANNLVSSHVRNRYALWIAQWNTHCDYQGSYGLWQYSSSGSVPGVAG |
| | RVDMDYAYVDYPSIIKNAGLNGCKNGGSDQAARTSSIDEVAREVINGAWGNGSTRKQRLT |
| | SAGYDYASVAK |
| SEQ ID NO: 51 | MKNWETCDADEIKLLTTHYTKGRQGLKINKIVLHHNAGNLSIQDCYNVWQTREASAHYQV |
| (CCB3.1.1) | QSDGRIGQLVWDYDTAWHAGNWNANATSIGIEHADISTSPWQISAQCLENGAHLVAALCR |
| | LYGLGRPTWMKNVFPHSYFYATACPCSIRDSQNSDYMKRAGEWYDAMTGAVKQQDQTKSG |
| | EEYPMIKSEIVVDGDVSSVEIHVPWGVNNSVGMTLTRAGNVVTANGAGGIKAGDAQWAKA |
| | NEYIPEGFRPTSLATITLTGGRAALLVQPDGSIYYDGDARDCTTHISGAWITKDNQPK |
| SEQ ID NO: 52 | MKNWETCDADEIKLLTTHYTKGRQGLKINKIVLHHNAGNLSIQDCYNVWQTREASAHYQV |
| (CCB3.1.2) | QSDGRIGQLVWDYDTAWHAGNWNANATSIGIEHADISTSPWQISAQCLENGAHLVAALCK |
| | LYGLGRPTWMKNVFPHSYFYATACPCSIRDSQNSDYMKRAGDWYDAMTGVVKQQNQTKSG |
| | EEYPMIKSEIVVDGNVSSVEIHVPWGVNNSVGMTLTRAGNVVTANGAGGIKAGDAQWAKA |
| | NETIPEGFRPTSLSTITLTGGRAALLVQPDGSMYYDGDARDCTTHLSGTWITTDNQPK |
| SEQ ID NO: 53 | MRNWETCDADEIKLLTKHYTKGRQGLKINKIVLHHNAGNLSIQDCYNVWQTREASAHYQV |
| (CCB3.1.3) | QSDGRVGQLVWDYDTAWHAGNWRANATSIGIEHADISTSPWQISAQCLENGAHLVAALCK |
| | LYGLGRPTWMKNVFPHSYFYATACPCSIRDSQNSDYMKRAGDWYDAITGVIKPQNQTRTM |
| | EEYPMIKSEIVVDGDVSSVEIHVPWGVNNSIGMTLTRTGNVVTANGAGGIKAGDAQWAKA |
| | NETIPEGFRPTSLATIALTGGRGAFLVQPDGSIYYDGDARDCTTHISGAWVTKDNQPK |
| SEQ ID NO: 54 | MALNGIDISWYQRGINIAEIPADFVIVKATEGTGYINPCFRTQADATLNSGKLLGIYHYI |
| (CCB4.1.1) | SGGSWQAEAEYFVNTVKDYVGRAVLALDFESGYNSAYGDTAYLQQCAQTVYNLTGVRPLL |
| | YGSQRDYGRLAAVSNATNCGLWIAQYKNYAHIGYQDTPWNEDAYSCAIRQYSSAGALPNY |
| | GGNLDLNKFYGDATAWQSYARSDKQTQEQQAPQVPAVSEVTHDGDISTVYVHVPWAVSQD |
| | IRMAITRVGNVVTVNGCGGIHAGDAHWAKANEYIPEGFKPTVLSTITLTGGRAAILVQPD |
| | GSMYYDGDARNCTTHLNGAWVTNDNQPE |
| SEQ ID NO: 55 | MALNGIDISWYQRGINIAAVPADFVIVKATEGTGYTNPCFREQADATLNSGKLLGIYHYI |
| (CCB4.1.2) | SGGNWQAEAEYFVNTVKDYVGRAVLALDFESGGNSAYDDIAYLQQCAQAVYNLTGVRTLL |
| | YGGQRDYGRLAAVSKATNCGLWIAQYRDYAHIGYQNAPWNEGAYECAIRQYSSSGALPNY |
| | GGNLDLNKFYGDRAAWESYARSDRQQAQEEQPMPAISEITHDGDVSTVYVHIPWSVQQDI |
| | RMTVVRVGNVVTVNGCGGMNAGDAQWAKANEYIPDGFKPTVLSTITLTGGRGALLVQPDG |
| | SIYYDGDARNCTTHLSGAWVTKDNQPE |
| SEQ ID NO: 56 | MALNGIDISWYQRGINIAAVPADFVIVKATEGTGYINPCFREQADATLNSGKLLGIYHYI |
| (CCB4.1.3) | SGGNWQAEAQYFVNTVKDYVGRAVLALDFESGGNSAYGDTAYLQQCAQTVYNLTGVHPLL |
| | YGSQRDYGSLAAVGNATNCGLWIAQYPNYARTGYQNTPWNEGAYSCAMRQYSSSGALPGY |
| | GGNLDLDKFYGDAAAWQAYAKSDKQQTVEEQEPMASAIAHDGDISSFTLHIPWGTDAKQV |
| | MRFARHGNVVTVNGCGFVASGGGSWVKAWEPVLEGFRPTTLATIQISGGGNSSLMVFPDG |
| | SICWDGDAFNGFAHVNGAWITNDNQPK |
| SEQ ID NO: 57 | MALNGIDVSWYQRGINIAAVPADFVIVKATEGAWYTNPCFHAQADATLNSGKLLGIYHYI |
| (CCB4.1.4) | SGGNAQAEMQYFVNAVKPYIGRAILALDFESGSNSAYGDTAYLQQCAQTVYNLTGVRPLL |
| | YGSQRDYGRLAAVSKATNCGLWIAQYANNDHTGYQDKPWNEDAYGCAIRQYSSAGALPNY |
| | GGNLDLNKFYGDRTAWNKYAQSDHATPPPAPKPDVSPIEHDGDISSFTMHIPWGTNQDQR |
| | MVFTRCGNVVTVNGCGCVVCGGGSWVKAREQVPDGFRPISLATIRLSGNGTGSIMVKPDG |
| | SICWDGDGKNCFTHISGTWITKDNQPK |
| SEQ ID NO: 58 | MALNGIDVSWYQRGINIAAVPADFVIVKATEGAWYTNPCFHAQADATLNSGKLLGIYHYI |
| (CCB4.1.5) | SGGNAQAEMQYFVNAVKPYIGRAILALDFESGSNSAYGDTAYLQQCAQTVYNLTGVRPLL |
| | YGSQCDYGRLARVSKATNCGLWIAQYANSAHTGYQSEPWNEGSYSCAIRQYSSAGALPNY |
| | GGNLDLNKFYGDRTAWNKYAQSDHATPPPAPKPAPKPDVSPIEHDGDISSFTMHIPWGTN |
| | QDQRMGITRCGNVVTVNGCGCVVCGGGSWIHAREQVPEGFRPVSLATIRLSGNGTGSIMV |
| | KPDGSICWDGDGKNCFTHINATWITKDNQPK |
| SEQ ID NO: 59 | MALNGIDVSWYQRGINIAAVPADFVIVKATEGAWYTNPCFHAQADATLNSGKLLGIYHYI |
| (CCB4.1.6) | SGGNAQAEMQYFVNAVKPYIGRAILALDFESGSNSAYTNTGYLQECAMKVYALTGVRPLL |
| | YGSQRDYGRLAQVSKATNCGLWIAQYPNSARTGYQNTPWNEDAYSCAIRQYSSAGALPNY |
| | GGNLDLNKFYGDRTAWNKYAQSDHATPPPAPKPDVSPIEHDGDISSFTMHIPWGTNQDQR |
| | MVFTRCGNVVTVNGCGCVVCGGGSWVKAREQVPDGFRPISLATIRLSGNGTGSIMVKPDG |
| | SICWDGDGKNCFTHASGAWITKDSQPK |
| SEQ ID NO: 60 | MALNGIDISWYQRGINIAAVPADFVIVKATEGTWYTNPCFRTQADATLNSGKLLGIYHYI |
| (CCB4.1.7) | NGGNAKAEAQYFVNAAKPYIGRAVLALDFESGSNSAYKDAGYLQVCATEVYALTGVRPLL |
| | YGSQCDYGRLAQVSKATNCGLWIAQYANNNHTGYQNKPWNEDSYGCAIRQYSSAGALPNY |
| | GGNLDLNKFYGDRAAWNKYAQSDHATPPPAPKPAPKPATSPIVKDGDISGFNMHIPWGTN |
| | DGQIMHFTRCGNVVTVNGCGYVKCVGGSWLKAGERVPEGFRPVTLSTIHLSGNGSGSLMV |
| | KPDGSIYWDGTWKENFTHINAAWITKDNQPK |
| SEQ ID NO: 61 | MPLKVVDVYSGSPRWYATDDNADAVIIKATQGTGYVNPFCDIDYQSAKKAGKLLGVYHYA |
| (CCB7.1.1) | SGGDPISEANYFLKNVKGYIHEAILGLDWESAQNASWGNANWSRQFVNEVHRQTGVWPII |
| | YVQFSAVWQVANCADTCGLWGAGYPWYTNSWMVPPFLSSYNFAPWKALTGWQFTGNTEDR |
| | SLFYVDANGWKAIAKGDGSITHTAQPAPQENEVKTSNVPSYAGTSWQDSLGVTWHAENGK |
| | FTANTDLHLRWGATPSSSVISVLHAGDVVKYDAWARTNGFVYVRQPRGNGQYGYVAVRNA |
| | YTNEAYGKFE |
| SEQ ID NO: 62 | MSKKGIDVSVWQGDIDFNSVKASGVEFVIIRAGYGTEHKDKWFEENYRKAKATGLDVGSY |
| (CCB2M80) | WYSYASSAGEASLEAQSLANTLSGKSFEYPIYFDLEEKSQLNRGRDFCDSLITGFCNKLE |
| | TCGYYAGFYTSLSTANNLVPSHVRDRYALWIAQWNTHCSYEGSYGLWQYSSSGSVSGIVG |
| | RVDMDYTYKDYPNVIKKAGLNGYQNGESHQAPQISSIDEVAREVINGDWGNGSTRKNRLI |
| | SAGYDYTSVQNKVNKLLGVKTRRKSVDELAREVIRGTWGNGSTRKNRLTSAGYDYNAVQK |
| | RVDELL |
| SEQ ID NO: 63 | MSKKGIDVSEWQGDIDFNAVKASGVEFVIIRAGYGIGCKDKWFEQNYRKAKTCGLDVGAY |
| (CCB230a) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTSEIVVDGNVSSVEIHVPWGVNNSVGM |
| | TLTRAGNVVTANGAGGIKAGDAQWAKANETIPEGFRPTSLSTITLTGGRAALLVQPDGSM |
| | YYDGDARDCTTHLSGTWITNDNQPE |
| SEQ ID NO: 64 | MSKKGIDVSEWQGDIDFNAVKASGVEFVIIRAGYGIGCKDKWFEQNYRKAKTCGLDVGAY |
| (CCB230b) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTYDAMTGVVKQQNQTKSGEEYPMIKSE |
| | IVVDGNVSSVEIHVPWGVNNSVGMTLTRAGNVVTANGAGGIKAGDAQWAKANETIPEGFR |
| | PTSLSTITLTGGRAALLVQPDGSMYYDGDARDCTTHLSGTWITNDNQPE |
| SEQ ID NO: 65 | MSKKGIDVSEWQGDIDFNAVKASGVEFVIIRAGYGIGCKDKWFEQNYRKAKTCGLDVGAY |
| (CCB240) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTSEITHDGDISTVYVHIPWSVQQDIRM |
| | AVVRVGNVVTVNGCGGMSAGDAQWAKANETIPEGFRPTSLSTITLTGGRAALLVQPDGSI |
| | YYDGDARDCTTHISGVWITKDTQPK |
| SEQ ID NO: 66 | MSKKGIDVSEWQGDIDFNAVKASGVEFVIIRAGYGIGCKDKWFEQNYRKAKTCGLDVGAY |
| (CCB260) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTIANPSGSKDGWQGSGDTWQYFENGSA |
| | VKNDWRKVDNCWYYLDESGNAVTGWKQINNHWYYFDTKHDGSFGTALTGWQQINGKKYYF |
| | DLQNAWMLTGKQTIDGKLYTFGDDGALQEATTQETKPAEAKPSIDKQIVLDDQLKAYIQS |
| | EIKAQLAKLKIKMEE |
| SEQ ID NO: 67 | MSKKGIDVSEWQGDIDFNAVKASGVEFVIIRAGYGIGCKDKWFEQNYRKAKTCGLDVGAY |
| (CCB270) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTQAIAKGDGTVTHTAQPAPQKNEAKTS |
| | NVPSYAGTTWQDSLGVTWHAENGKFTANTDLHLRWGATPSSSVISVLHAGDVVKYDAWAR |
| | TNGFVYIRQPRGNGQYGYVAVRNAYTNEAYGKFE |
| SEQ ID NO: 68 | MSKKGIDVSEWQGDIDFNAVKASGVEFVIIRAGYGIGCKDKWFEQNYRKAKTCGLDVGAY |
| (CCB280) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTQRLANPTGQVSSRPLIFGDSSASSIS |
| | NNETWTDNLGDTWHREVGRFTSSRPLTLRWGALPTSTAIAELPAGSVIDYDAWSRHDGEV |
| | WIRQPRGNGQYGYLPCRNAVTNEAYGTFSE |
| SEQ ID NO: 69 | MSKKGIDVSVWQGDIDFNSVKASGVEFVIIRAGYGIGCKDKWFEENYRKAKATGLDVGSY |
| (CCB2M81_7) | WYSYASSAGEASLEAQSLANTLSGKSFEYPIYFDLEEKSQLNRGRDFCDSLITGFCNKLE |
| | TCGYYAGFYTSLSTANNLVPSHVRDRYALWIAQWNTHCSYEGSYGLWQYSSSGSVSGIVG |
| | RVDMDYTYKDYPNVIKKAGLNGYQNGESHQAPQISSIDEVAREVINGDWGNGSTRKNRLI |
| | SAGYDYTSVQNKVNKLLGVKACRKSVDELAREVIRGTWGNGSTRKNRLTSAGYDYNAVQK |
| | RVNELL |
| SEQ ID NO: 70 | MSKKGIDVSVWQGDIDFNSVKASGVEFVIIRAGYGIGCKDKWFEENYRKAKATGLDVGSY |
| (CCB2M83_6) | WYSYANSSSEAAEEAQSCVNMLSGKSFEYPIYFDLEEKSQLNRGRDFCDSLITGFCNKLE |
| | TCGYYAGFYTSLSTANNLVPSHVRDRYALWIAQWNTHCSYEGSYGLWQYSSSGSVSGIVG |
| | RVDMDYTYKDYPNVIKKAGLNGYQNGESYTAPQTSSIDEVAREVINGDWGNGSTRKNRLI |
| | SAGYDYTSVQNKVNKLLGVKACRKSVDELAREVIRGTWGNGSTRKNRLTSAGYDYNAVQK |
| | RVNELL |
| SEQ ID NO: 71 | MSKKGIDVSVWQGDIDFNSVKASGVEFVIIRAGYGIGCKDKWFEENYRKAKATGLDVGSY |
| (CCB2M84_97) | WYSYANSSSEAAEEAQSCVNMLSGKSFEYPIYFDLEEKSQLNRGRDFCDSLITGFCNKLE |
| | TCGYYAGFYTSLSTANNLVSSHVRNRYALWIAQWNTHCSYEGSYGLWQYSSSGSVSGIVG |
| | RVDMDYAYVDYPNVIKKAGLNGYQNGESYTAPQTSSIDEVAREVINGDWGNGSTRKNRLI |
| | SAGYDYTSVQNKVNKLLGVKACRKSVDELAREVIRGTWGNGSTRKNRLTSAGYDYNAVQK |
| | RVNELL |
| SEQ ID NO: 72 | MSKKGIDVSEWQGDIDFNAVKASGVEFVIIRAGYGTGCKDKWFEQNYRKAKTVGLDVGAY |
| (CCB2M94_8) | WYSYASSAGEASEEAQSCVNMLSGKSFEYPIYFDLEEKSQLNRGRAFCDSLITSFCSKLE |
| | TYGYYAGFYTSLSTANNLVSSHVRNRYALWIAQWNTHCNYQGSYGLWQYSSNGSVPGVAG |
| | RVDMDYAYKDYPSIIKNAGLNGYKNGGSDQAARTSSIDEVAREVINGAWGNGNERKQRLT |
| | SAGYDYASVQNKVNELLGVKAYRKSVDELAREVIRGTWGNGNERKQRLTQAGYDYATVQK |
| | RVNELL |
| SEQ ID NO: 73 | MSKKGIDVSEWQGDIDFNAVKASGVEFVIIRAGYGIGCKDKWFEQNYRKAKTCGLDVGAY |
| (CCB2.2) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAARTSSIDEVAREVINGAWGNGKERKQRLT |
| | QAGYDYASVQNKVNELLG |
| SEQ ID NO: 74 | MSKKGIDVSEWQGDIDFNAVKASGVEFVIIRAGYGIGCKDKWFEQNYRKAKTCGLDVGAY |
| (CCB2.3) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVD |
| SEQ ID NO: 75 | MSKKGIDVSEWQGDIDFNAVKASGVEFVIIRAGYGIGCKDKWFEQNYRKAKTCGLDVGAY |
| (CCB2.4) | WYSYANSGFEAAEEAQSCVNMLSGKSFEYPVYFDLEEKSQLNRGRAFCDSLITSFCSKLE |
| | TYGYYAGFYTSLSVANNLVSAHVRNRYALWIAQWNTHCNYQGSYGLWQYSSSGSVPGVAG |
| | RVDMDYAYVDYPSIIKNAGLNGCKNGGSDQAART |
| SEQ ID NO: 76 | MKNWETCDADEIKLLTTHYTKGRQGLKINKIVLHHNAGNLSIQDCYNVWQTREASAHYQV |
| (CCB3.2) | QSDGRIGQLVWDYDTAWHAGNWNANATSIGIEHADISTSPWQISAQCLENGAHLVAALCK |
| | LYGLGRPTWMKNVFPHSYFYATACPCSIRDSQNLDYMKRAGDWYDAMTGVVKQQNQTKSG |
| | EEYPMIK |
| SEQ ID NO: 77 | MALNGIDISSYQSSINIAAVPADFVIVKATEGTGYINPCFRAHADTILNSGKLLGIYHYI |
| (CCB4.2) | SGSGWQAEAEYFVNTVKDYIGRAVLALDFESGGNSAYGDTAYLQQCAQTVYNLTGVHPLI |
| | YGSQRDYGRLAAVSKATNCGLWVAQYANNNHTGYQNKPWNEDAYDCAIRQYSSSGSLPNY |
| | GGNLDLNKFYGDAAAWQSYAKSDNQTQQSQEAQQAE |

Preferably, the polypeptides of the invention can comprise or consist of an amino acid sequence as set forth in SEQ ID No: 22 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence as set forth in SEQ ID No: 23 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence as set forth in SEQ ID No: 24 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence as set forth in SEQ ID No: 25 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence as set forth in SEQ ID No: 26 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 51 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 52 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 53 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 54 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 55 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 56 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 57 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 58 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 59 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 60 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 61 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 61 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 62 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 63 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 64 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 65 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 66 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 67 or a sequence having at least 80, 85, 90, 95, 96, 97, 98, 99, 99.5 or 100% amino acid sequence identity thereto, or a fragment thereof.

Preferably the polypeptides of the invention can comprise or consist of an amino acid sequence as set forth in SEQ ID No: 68 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence as set forth in SEQ ID No: 76 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence as set forth in SEQ ID No: 77 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence selected from the group consisting of: SEQ ID No: 21, 23, 25, 26, 73, 74. 75, 76 and 77 or a sequence having at least 80, 85, 90, 95, 96, 97, 98, 99, 99.5 or 100% amino acid sequence identity thereto, or a fragment thereof.

Preferably, the polypeptides of the invention can comprise or consist of an amino acid sequence selected from the group consisting of: SEQ ID No: 23, 25, 26, 76 and 77 or a sequence having at least 80, 85, 90, 95, 96, 97, 98, 99, 99.5 or 100% amino acid sequence identity thereto, or a fragment thereof.

The domains and sequences described herein can be used in a plug-and-play fashion to alter and/or improve one or more polypeptide activity as described herein, such as endolysin activity, peptidoglycan substrate selectivity, and/or polypeptide solubility.

In some embodiments, polypeptides of the invention can comprise or consist of the following formula, in an N-terminal to C-terminal or C-terminal to N-terminal direction:

R1ₐ-Xₙ

Where R1 is a hydrolase domain as described herein or an amino acid sequence of Table 1 or a sequence having at least 80% identity thereto, or a fragment thereof, and a is an integer from 1-3, preferably 1; and X is a linker as described herein or an amino acid sequence of Table 3 or a sequence having at least 80% identity thereto, or a fragment thereof, and n is an integer from 0-10, preferably 1 or 2. Preferably, polypeptides of the invention can have the above formula in the N-terminal to C-terminal direction.

In some embodiments, polypeptides of the invention can comprise or consist of the following formula, in an N-terminal to C-terminal or C-terminal to N-terminal direction:

R1ₐ-Xₙ—R2_{b}

Where R1 is a hydrolase domain as described herein or an amino acid sequence of Table 1 or a sequence having at least 80% identity thereto, or a fragment thereof, and a is an integer from 1-3, preferably 1; R2 is a cell wall binding domain as described herein or an amino acid sequence of Table 2 or a sequence having at least 80% identity thereto, or a fragment thereof, and b is an integer from 1-10, preferably 2; and X is a linker as described herein or an amino acid sequence of Table 3 or a sequence having at least 80% identity thereto, or a fragment thereof, and n is an integer from 0-10, preferably 1 or 2. Preferably, polypeptides of the invention can have the above formula in the N-terminal to C-terminal direction.

In some embodiments, polypeptides of the invention can comprise or consist of the following formula, in an N-terminal to C-terminal or C-terminal to N-terminal direction:

R1ₐ-Xₙ—R2_{b}-Yₘ—R3_{c}

Where R1 is a hydrolase domain as described herein or an amino acid sequence of Table 1 or a sequence having at least 80% identity thereto, or a fragment thereof, and a is an integer from 1-3, preferably 1; R2 is a cell wall binding domain as described herein or an amino acid sequence of Table 2 or a sequence having at least 80% identity thereto, or a fragment thereof, and b is an integer from 1-10, preferably 1 or 2; R3 is a hydrolase domain as described herein or an amino acid sequence of Table 1 or a sequence having at least 80% identity thereto, or a fragment thereof, or a cell wall binding domain as described herein or an amino acid sequence of Table 2 or a sequence having at least 80% identity thereto, or a fragment thereof, and c is an integer from 1-10, preferably 1; X is a linker as described herein or an amino acid sequence of Table 3 or a sequence having at least 80% identity thereto, or a fragment thereof, and n is an integer from 0-10, preferably 1 or 2; and Y is a linker as described herein or an amino acid sequence of Table 3 or a sequence having at least 80% identity thereto, or a fragment thereof, and m is an integer from 0-10, preferably 1 or 2. Preferably, polypeptides of the invention can have the above formula in the N-terminal to C-terminal direction.

Where R1, R2 and/or R3 comprises a plurality of domains or sequences as described herein, they can be the same or different. Where R1, R2 and/or R3 comprises a plurality of domains or sequences as described herein, a linker as described herein may be present between each domain or sequence.

Polypeptides of the invention can exhibit anti-bacterial activity, such as anti-bacterial activity against gram positive, gram negative and/or gram-variable bacteria, preferably anti-Gardnerella spp. activity, such as anti-bacterial activity against G. vaginalis, G. leopoldii, G. piotii, and/or G. swidsinskii, preferably G. vaginalis. Anti-bacterial activity can involve killing, stopping or reducing growth of and/or preventing re-growth of bacteria. Anti-bacterial activity can involve disrupting and/or preventing a biofilm, preferably a biofilm comprising Gardnerella spp., such as G. vaginalis, G. leopoldii, G. piotii, and/or G. swidsinskii, preferably G. vaginalis. Preferably, polypeptides of the invention exhibit anti-bacterial activity specifically against Gardnerella spp., such as G. vaginalis, G. leopoldii, G. piotii, and/or G. swidsinskii, preferably G. vaginalis. Preferably, polypeptides of the invention do not exhibit or exhibit a lower, preferably at least a 1-fold, 2-fold, 3-fold, 4-fold, 5-fold, 10-fold, 100-fold or 1000-fold lower anti-bacterial activity against bacteria other than Gardnerella spp., such as G. vaginalis, G. leopoldii, G. piotii, and/or G. swidsinskii, preferably G. vaginalis. Anti-bacterial activity can be measured by assays described herein and known to those skilled in the art.

Polypeptides of the invention can exhibit one or more endolysin polypeptide activity.

Polypeptides of the invention can exhibit hydrolase enzyme activity, preferably one or more activity that specifically modifies and/or cleaves chemical bonds in a substrate present in bacterial cell walls, preferably peptidoglycan, preferably Gardnerella spp. peptidoglycan, such as G. vaginalis, G. leopoldii, G. piotii, and/or G. swidsinskii peptidoglycan, preferably G. vaginalis peptidoglycan. Polypeptides of the invention can exhibit amidase, glycosidase, hydrolase and/or endo/carboxypeptidase enzyme activity, such as a muramidase, glucosaminidase, endopeptidase, and/or N-acetyl-muramoyl-L-alanine amidase enzyme activity. Preferably, polypeptides of the invention exhibit lytic activity causing lysis of bacterial cells, preferably lysis of Gardnerella spp. bacteria, such as G. vaginalis, G. leopoldii, G. piotii, and/or G. swidsinskii, preferably G. vaginalis. Preferably, polypeptides of the invention do not exhibit or exhibit a lower, preferably at least a 1-fold, 2-fold, 3-fold, 4-fold, 5-fold, 10-fold, 100-fold or 1000-fold lower hydrolase enzyme activity and/or lytic activity against bacteria other than Gardnerella spp., such as G. vaginalis, G. leopoldii, G. piotii, and/or G. swidsinskii, preferably G. vaginalis. Hydrolase enzyme activity and lytic activity can be measured by assays described herein and known to those skilled in the art.

Polypeptides of the invention can exhibit cell wall binding activity such that polypeptides interact with and/or bind to bacteria, bacterial cell walls and/or one or more specific substrate within bacterial cell walls. Preferably, polypeptides of the invention bind specifically to Gardnerella spp. bacteria, such as G. vaginalis, G. leopoldii, G. piotii, and/or G. swidsinskii, preferably G. vaginalis, their cell walls and/or a specific substrate within their cell walls, preferably peptidoglycan. Cell wall binding activity can be measured by assays described herein and known to those skilled in the art.

Polypeptides of the invention do not exhibit an activity as described herein, or exhibit a lower activity as described herein, such as at least a 1-fold, 2-fold, 3-fold, 4-fold, 5-fold, 10-fold, 100-fold or 1000-fold lower activity, against flora associated with a healthy vagina. Polypeptides of the invention do not exhibit an activity as described herein or exhibit a lower activity as described herein, such as at least a 1-fold, 2-fold, 3-fold, 4-fold, 5-fold, 10-fold, 100-fold or 1000-fold lower activity, against bacteria other than Gardnerella spp., such as G. vaginalis, G. leopoldii, G. piotii, and/or G. swidsinskii, preferably G. vaginalis. Preferably, polypeptides of the invention do not exhibit or exhibit a lower activity as described above against healthy vaginal commensal bacteria, such as Lactobacillus spp. including Lactobacillus crispatus, L. iners, L. jensenii, and L. gasseri.

Polypeptides of the invention exhibit a low or no observable resistance profile. Resistance of bacteria to anti-bacterial agents can be measured by assays described herein and known to those skilled in the art.

Polypeptides of the invention can exhibit one or more of the activities described herein, in particular such that the polypeptides are useful for preventing or treating a disease associated with a bacterial infection, such as BV.

Nucleic acids capable of encoding the polypeptide(s) of the invention are provided herein and constitute an aspect of the invention. Representative nucleic acid sequences in this context are polynucleotide sequences coding for the polypeptide of any of SEQ ID NO: 1 to 77, and sequences that hybridize, under stringent conditions, with complementary sequences of the DNA sequence(s). Further variants of these sequences and sequences of nucleic acids that hybridize with those sequences also are contemplated for use in production of polypeptides according to the disclosure, including natural variants that may be obtained. A large variety of isolated nucleic acid sequences or cDNA sequences that encode polypeptides of the invention and partial sequences that hybridize with such gene sequences are useful for recombinant production of the polypeptide(s) of the invention.

One skilled in the art will recognize that the DNA mutagenesis techniques described here and known in the art can produce a wide variety of DNA molecules that code for polypeptides of the invention yet that maintain the essential characteristics of the polypeptides described and provided herein.

Polypeptides or nucleic acids of the invention can be isolated, by which is meant that the polypeptides or nucleic acids will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Polypeptides and nucleic acid may be formulated with diluents or adjuvants and still for practical purposes be isolated—for example the polypeptides will normally be mixed with polymers or other carriers, or will be mixed with pharmaceutically acceptable carriers or diluents, when used in diagnosis or therapy.

Vectors comprising nucleic acids as described herein are provided and constitute an aspect of the invention. As is well known in the art, DNA sequences can be expressed by operatively linking them to an expression control sequence in an appropriate expression vector and employing that expression vector to transform an appropriate unicellular host. Such operative linking of a DNA sequence of this invention to an expression control sequence, of course, includes, if not already part of the DNA sequence, the provision of an initiation codon, ATG, in the correct reading frame upstream of the DNA sequence. A wide variety of host/expression vector combinations may be employed in expressing the DNA sequences of this invention. Useful expression vectors, for example, may consist of segments of chromosomal, non-chromosomal and synthetic DNA sequences. Any of a wide variety of expression control sequences—sequences that control the expression of a DNA sequence operatively linked to it—may be used in these vectors to express the DNA sequences of this invention.

A cell comprising a nucleic acid or vector as described herein is provided in accordance with another aspect of the invention. A wide variety of unicellular host cells are also useful in expressing the nucleic acid sequences of the invention. These hosts can include well known eukaryotic and prokaryotic hosts, such as strains of E. coli (e.g., BL21 DE3 and Arctic express), Lactobacillus spp., Lactococcus spp., Pseudomonas spp., Bacillus spp., Streptomyces spp., Clostridium spp., fungi such as yeasts (e.g. Saccharomyces cerevisiae), and animal cells (e.g., CHO, RI.1, B-W and L-M cells, African Green Monkey kidney cells (e.g., COS 1, COS 7, BSCI, BSC40, and 5 BMTIO), insect cells (e.g., Sf9), and human cells and plant cells in tissue culture. One skilled in the art will be able to select the proper vectors, expression control sequences, and hosts without undue experimentation to accomplish the desired expression without departing from the scope of the invention.

By “comprise” or “comprising” as used herein is meant to include, that is to say permitting the presence of one or more features or components. By “consists of” or “consisting of” is meant a product, for example a polypeptide as described herein having a defined number of amino acid residues which is not attached to a larger product. Those of skill in the art will appreciate that minor modifications to the N- or C-terminal of the polypeptide may be contemplated, such as the chemical modification of the terminal to add a protecting group or the like, e.g. the amidation of the C-terminus.

Polypeptides of the invention can comprise or consist of an amino acid sequence that is different to the amino acid sequences of the polypeptides described herein, but which retain the activity of polypeptides as described herein (“variants” of the polypeptides described herein). For example, polypeptides may have at least 80, 85, 90, 95, 96, 97, 98, 99 or 99.5% sequence identity with the amino acid sequences of polypeptides described herein and retain one or more activity of the polypeptides as described herein.

Percentage amino acid sequence identity with respect to the polypeptides described herein refers to the percentage of amino acid residues in a candidate sequence that are identical with the amino acid residues in the polypeptide sequence after aligning the sequences in the same reading frame and introducing gaps, if necessary, to achieve the maximum percent sequence identity, and not considering any conservative substitutions as part of the sequence identity. Determining the percent identity of two nucleotide or amino acid sequences can be performed using well-known techniques.

A fragment of a polypeptide as described herein includes polypeptides comprising amino acid sequences sufficiently identical to or derived from the amino acid sequence of the polypeptides of the disclosure, which include fewer amino acids than the full length polypeptide and exhibit at least one activity of the corresponding full-length polypeptide. Typically, activity of the full-length polypeptide is due to a biologically active portion comprising a domain or motif with at least one activity of the corresponding polypeptide. A biologically active portion of a polypeptide or polypeptide fragment of the disclosure can be a polypeptide which comprises or consists of 10, 25, 50, 100 of the full-length polypeptide. A fragment can comprise a polypeptide having 1, 2, 3, 4, 5, 6, 7, 8, 9 10, 15, 20, 25, 30, 35, 40, 45, 50, 75 or 100 fewer amino acids in length than the full-length polypeptide.

Mutations can be made in the amino acid sequences of the polypeptides described herein, or in the nucleic acid sequences encoding the polypeptides described herein, including the sequences set out in Tables 1 to 4 or variants or fragments thereof, such that a particular codon is changed to a codon which codes for a different amino acid, an amino acid is substituted for another amino acid, or one or more amino acids are deleted. Such a mutation is generally made by making the fewest amino acid or nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and activity of the resulting protein. A non-conservative change is more likely to alter the structure, activity or function of the resulting protein. The present invention should be considered to include sequences containing conservative changes which do not significantly alter the activity or binding characteristics of the resulting polypeptide.

Thus, one of skill in the art, based on a review of the amino acid sequences of the polypeptides described herein and their knowledge and the public information available for other endolysin polypeptides, can make amino acid changes or substitutions in the polypeptide sequences. Amino acid changes can be made to replace or substitute one or more, such as 1, 2, 3, 4, 5, 6, 7, 8, 9, 10, 15, amino acids in the sequence of the polypeptides provided herein to generate mutants or variants thereof. Such mutants or variants thereof may be predicted or tested for one or more activity of the polypeptides described herein. Changes can be made to the sequences of the polypeptides described herein, and such mutants or variants can be tested using the assays and methods described and exemplified herein, including in the examples. One of skill in the art, on the basis of the domain structure of the polypeptides, can predict one or more, one or several amino acids suitable for substitution or replacement and/or one or more amino acids which are not suitable for substitution or replacement, including reasonable conservative or non-conservative substitutions.

Polypeptides of the invention can comprise a “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

Polypeptides of the invention can be operably linked to another polypeptide (hereinafter referred to as “chimeric” or “fusion” proteins), for example a heterologous polypeptide. Such fusion proteins can be produced using techniques known in the art by combining two or more polypeptides having active sites. Fusion proteins can have one or more activity of the polypeptides described herein. Fusion protein polypeptides can act independently on the same target molecule, preferably peptidoglycan, specifically Gardnerella spp. peptidoglycan, thus providing a polypeptide having an improvement in one or more activity as described herein. Alternatively, fusion protein polypeptides can act independently on different target molecules, one of which is preferably peptidoglycan, specifically Gardnerella spp. peptidoglycan, thus providing a polypeptide having one or more activity in addition to the polypeptides described herein, for example, targeting different bacteria, for example to treat two or more different bacterial infections at the same time. By “operably linked” is meant that the polypeptide of the disclosure and heterologous polypeptide, for example, are fused in-frame. The heterologous polypeptide can be fused to the N-terminus or C-terminus of the polypeptide of the disclosure. Chimeric proteins are produced enzymatically by chemical synthesis, or by recombinant DNA technology. A number of chimeric endolysin polypeptides have been produced and studied. One example of a useful fusion protein is a GST fusion protein in which the polypeptide of the disclosure is fused to the C-terminus of a GST. Such a chimeric protein can facilitate the purification of a recombinant polypeptide of the disclosure. Polypeptides of the invention can contain a heterologous signal sequence at its N-terminus. For example, the native signal sequence of a polypeptide of the disclosure can be removed and replaced with a signal sequence from another known protein.

Signal sequence, solubility tags and other additions/modifications familiar to those skilled in the art may be added to a polypeptide to facilitate transmembrane movement of the protein and peptides and peptide fragments of the disclosure to and from mucous membranes, as well as by facilitating secretion and isolation of the secreted protein or other proteins of interest. Signal sequences are typically characterised by a core of hydrophobic amino acids which are generally cleaved from the mature protein during secretion in one or more cleavage events and are well characterised in the art, and facilitate separation, isolation and/or purification, from for example, any suitable medium by art-recognised methods.

A fusion protein can comprise a polypeptide as described herein and a protein or polypeptide having a different capability, or providing an additional capability or added character to the polypeptide. The fusion protein may comprise an immunoglobulin polypeptide in which all or part of a polypeptide of the disclosure is fused to sequences derived from a member of the immunoglobulin protein family. The immunoglobulin may be an antibody, for example an antibody directed to a surface protein or epitope of a Gardnerella spp. bacteria, such as G. vaginalis, G. leopoldii, G. piotii, and G. swidsinskii, preferably G. vaginalis.

Polypeptides of the invention can contain one or more additional anti-bacterial agent conjugated at the N- or C-terminal to form fusion proteins including commonly known antimicrobial peptides, antibodies/antibody derivatives or small molecules with activity against Gardnerella spp., preferably G. vaginalis, G. leopoldii, G. piotii, and G. swidsinskii.

Chimeric and fusion proteins and peptides can be produced by standard molecular biology, and synthetic biology approaches and can comprise any of the polypeptide sequences described herein, in any combination, orientation or manifestations e.g. truncations/mutations/amino acid substitutions. These chimeric and fusion proteins and peptides include the derivatives described in the above paragraphs for the purposes of facilitating purification, manufacture, formulation, enhancing potency, selectivity, solubility, bioavailability etc.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Polypeptides of the invention can be provided as compositions, for example a pharmaceutical composition comprising a polypeptide as described herein, or nucleic acids, vectors or cells as described herein, and at least one pharmaceutically acceptable excipient. Therapeutic or pharmaceutical compositions may comprise one or more polypeptide(s) of the invention, and optionally one or more natural, truncated, chimeric or shuffled endolysin polypeptide, optionally combined with other components such as a carrier, vehicle, polypeptide, polynucleotide, holin protein(s), one or more antibiotics or suitable excipients, carriers or vehicles.

The invention provides polypeptides, nucleic acids, vectors and cells as described herein and therapeutic compositions or pharmaceutical compositions comprising the same for use in preventing or treating a bacterial infection, for example by killing or preventing growth of gram-variable bacteria, specifically Gardnerella spp. bacteria such as G. vaginalis, G. leopoldii, G. piotii, and G. swidsinskii, preferably G. vaginalis. Compositions are thereby provided for therapeutic applications and local or systemic administration. Compositions can comprise other therapeutic and/or prophylactic ingredients.

Pharmaceutical compositions of the invention can be administered such that a therapeutically effective amount of the polypeptide of the invention is delivered and by any of the accepted modes of administration for agents that serve similar utilities. Pharmaceutical compositions include those suitable for oral or topical administration, preferably topical administration, preferably vaginal administration.

Pharmaceutical compositions of the invention can be prepared with one or more conventional adjuvants, carriers, or diluents and placed into dosage forms, such as unit dosages. The pharmaceutical compositions and dosage forms can be comprised of conventional ingredients in conventional proportions and the dosage forms can contain any suitable effective amount of the polypeptide commensurate with the intended daily dosage range to be employed.

Pharmaceutical compositions may take any of a number of different forms depending, in particular, on the manner in which it is to be used. Thus, for example, the agent or composition may be in the form of a powder, tablet, capsule, liquid, cream, gel, lotion, hydrogel, foam, micellar solution, liposome suspension or any other suitable form that may be administered to a person or animal in need of treatment. It will be appreciated that the carrier of the pharmaceutical composition according to the invention should be one which is well-tolerated by the subject to whom it is given. Preferably, pharmaceutical compositions can be formulated as a tablet or capsule for oral administration or a cream, gel, lotion, pessary or vaginal suppository for topical administration. In some embodiments, pharmaceutical compositions can include a vaginal pad or tampon, wherein a liquid, cream, gel or lotion, for example, can be applied to the pad or tampon.

A “pharmaceutically acceptable carrier” as referred to herein, is any known compound or combination of known compounds that are known to those skilled in the art to be useful in formulating pharmaceutical compositions.

Polypeptides, nucleic acids, vectors and cells and pharmaceutical compositions as described herein can be used in a monotherapy (i.e. use of the polypeptide alone) for preventing or treating a bacterial infection in a subject, preferably BV. Alternatively, the polypeptide may be used as an adjunct to, or in combination with, one or more additional active agents including: A polypeptide, nucleic acid, vector, cell or composition as described herein can be administered in combination with one or more additional therapeutic agent. Administration includes administration of a formulation that includes the polypeptide, nucleic acid, vector, cell or composition and one or more additional therapeutic agent, or the essentially simultaneous, sequential or separate administration of separate formulations of the polypeptide, nucleic acid, vector, cell or composition and one or more additional therapeutic agents.

Additional therapeutic agents can include one or more antimicrobial agent and/or one or more conventional antibiotic. In order to accelerate treatment of infection, the additional therapeutic agent may include an agent that can potentiate the anti-bacterial or bactericidal activity of the polypeptide. Antimicrobials act largely by interfering with the structure or function of a bacterial cell by inhibition of cell wall synthesis, inhibition of cell-membrane function and/or inhibition of metabolic functions, including protein and DNA synthesis. Antibiotics can be subgrouped broadly into those affecting cell wall peptidoglycan biosynthesis and those affecting DNA or protein synthesis in gram positive bacteria. Cell wall synthesis inhibitors, including penicillin and antibiotics like it, disrupt the rigid outer cell wall so that the relatively unsupported cell swells and eventually ruptures. Antibiotics affecting cell wall peptidoglycan biosynthesis include glycopeptides, which inhibit peptidoglycan synthesis by preventing the incorporation of N-acetylmuramic acid (NAM) and N-acetylglucosamine (NAG) peptide subunits into the peptidoglycan matrix. Available glycopeptides include vancomycin and teicoplanin. Penicillins act by inhibiting the formation of peptidoglycan cross-links. The functional group of penicillins, the beta-lactam moiety, binds and inhibits D,D-transpeptidase that links the peptidoglycan molecules in bacteria. Hydrolytic enzymes continue to break down the cell wall, causing cytolysis or death due to osmotic pressure. Common penicillins include oxacillin, ampicillin and cloxacillin. Polypeptides interfere with the dephosphorylation of the C55-isoprenyl pyrophosphate, a molecule that carries peptidoglycan building-blocks outside of the plasma membrane. A cell wall-impacting polypeptide is bacitracin.

Preferably, a polypeptide, nucleic acid, vector, cell or composition as described herein can be administered in combination with one or more agent conventionally utilised in the treatment of BV. Agents conventionally utilised in the treatment of BV include antibiotics interfering with DNA synthesis, such as nitroimidazoles, preferably selected from metronidazole and secnidazole; inhibitors of bacterial protein synthesis such as clindamycin; non-antibiotic therapies such as acidification through short chain organic acids (e.g. acetic, lactic), and dendrimer-based therapies. Preferably, a polypeptide, nucleic acid, vector, cell or composition as described herein can be administered in combination with metronidazole or secnidazole, optionally in combination with clindamycin, preferably with metronidazole or metronidazole and clindamycin.

The invention also encompasses use of at least one amino acid sequence selected from SEQ ID NOs: 1-6 or a sequence having at least 80, 85, 90, 95, 96, 97, 98, 99, 99.5 or 100% identity thereto or a fragment thereof; and/or at least one amino acid sequence selected from SEQ ID NOs: 7-13 or a sequence having at least 80, 85, 90, 95, 96, 97, 98, 99, 99.5 or 100% identity thereto or a fragment thereof to create a chimeric polypeptide having anti-Gardnerella spp. activity.

Preferably, the invention encompasses use of at least one amino acid sequence selected from SEQ ID NOs: 2-6 or a sequence having at least 80, 85, 90, 95, 96, 97, 98, 99, 99.5 or 100% identity thereto or a fragment thereof; and/or at least one amino acid sequence selected from SEQ ID NOs: 9-13 or a sequence having at least 80, 85, 90, 95, 96, 97, 98, 99, 99.5 or 100% identity thereto or a fragment thereof to create a chimeric polypeptide having anti-Gardnerella spp. activity.

The invention also encompasses a method of treating or preventing a bacterial infection in a subject, preferably a bacterial infection caused by Gardnerella spp. bacteria, such as G. vaginalis, G. leopoldii, G. piotii, and/or G. swidsinskii, preferably G. vaginalis; preferably bacterial vaginosis (BV), and a polypeptide, nucleic acid, vector, cell or composition as described herein for use in such methods.

The invention also encompasses a method of preventing or reducing the risk of recurrent BV infection. The invention also encompasses a method of reducing the incidence and/or risk of a preterm birth in a subject. The invention also encompasses a method of reducing the incidence and/or risk of a subject transmitting a sexually transmitted infection (STI) to another person. The invention also encompasses a method of preventing or treating any vaginal infections which are related to a dysbiotic vaginal microbiome. The invention also encompasses a method of preventing or treating any vaginal dysbiosis or infection which may impact fertility or have an affect on In Vitro Fertilisation (IVF) outcomes. The invention also encompasses a method of improving fertility. The invention also encompasses a method of treating infertility. The invention also encompasses a method of preventing or treating any vaginal infections which are related to a dysbiotic vaginal microbiome.

A method of treating or preventing comprises administering a polypeptide, nucleic acid, vector, cell or composition as described herein to a subject for the purposes of ameliorating a disease, disorder or condition (i.e., slowing or arresting or reducing the development of the disease, disorder or condition or at least one of the clinical symptoms thereof); alleviating or ameliorating at least one physical parameter including those which may not be discernible by the patient; modulating the disease, disorder or condition, either physically, (e.g., stabilization of a discernible symptom), physiologically, (e.g., stabilization of a physical parameter), or both; or preventing or delaying the onset or development or progression of the disease or disorder or a clinical symptom thereof.

A subject is in need of a treatment if the subject would benefit biologically, medically or in quality of life from such treatment. Treatment will typically be carried out by a physician who will administer a therapeutically effective amount of the polypeptide, nucleic acid, vector, cells or composition as described herein. Suitably the subject is a human, preferably a human female. Preferably, the subject is a human female presenting with an off-white (milky or gray), thin, homogeneous vaginal discharge, vaginal pH greater than or equal to 4.7, presence of clue cells of greater than or equal to 20% of the total epithelial cells on microscopic examination of a vaginal saline wet mount, a positive 10% KOH Whiff test, a gram stain slide Nugent score equal to, or higher than four on bacterial analysis of vaginal samples, Hay-Ison criteria or a combination thereof. In some embodiments, BV is confirmed by the presence of four Amsel criteria parameters, Hay-Ison criteria for BV and presence of symptoms: thin grey white vaginal discharge and foul fishy odor (acute symptoms).

Alternatively, a subject may have G. vaginalis within the vaginal flora but lack presentation of acute BV related symptoms, with Nugent scores and Hay-Ison criteria which do describe acute BV, or display Nugent scores and/or Hay-Ison criteria which portend the imminent growth of G. vaginalis, often described as intermediate flora In these asymptomatic cases, G. vaginalis presence may still result in a 10-fold higher risk of miscarriage, a 2-fold increased risk of preterm birth and statistically increased risk of STI transmission. A polypeptide of the invention, nucleic acid, vector, cell or composition as described herein can therefore be administered prophylactically to prevent or reduce the risk of prenatal health concerns, increase success rates of IVF cycles, and/or to reduce the spread of STIs.

A therapeutically effective amount of a polypeptide, nucleic acid, vector, cells or composition as described herein refers to an amount that will be effective for the treatment described above, for example slowing, arresting, reducing or preventing the disease, disorder or condition or symptom thereof. Typically, a subject in need thereof is a subject presenting symptoms of the disease, disorder or condition. Alternatively, a subject may be susceptible to the disease, disorder or condition or has been tested for the disease, disorder or condition but has not yet shown symptoms.

In one aspect, the bacterial infection can be characterised by a biofilm and the invention can provide a method for the prevention, dispersion, treatment and/or decolonization of a bacterial biofilm and the prevention of infections after dispersion of biofilm(s) wherein the biofilm comprises Gardnerella spp. bacteria, such as G. vaginalis, G. leopoldii, G. piotii, and/or G. swidsinskii, preferably G. vaginalis.

A polypeptide, nucleic acid, vector, cells or composition as described herein can be administered locally or systemically, preferably vaginally or orally, preferably vaginally. The therapeutically effective dose will be estimated initially either in cell culture assays or in animal models, usually in mice, rabbits, dogs, or pigs. The animal model is also used to achieve a desirable concentration range and route of administration. Such information can then be used as a foundation to determine useful doses and routes for administration in humans through clinical studies.

Treatment duration and posology will be selected by the physician in part based upon these scientifically robust clinical studies, but also on a number of factors, including but not limited to: severity of BV and associated conditions as scored by the Nugent score/Amstel criteria, the weight of the patient, co-morbidities, diet, desired duration of treatment, method of administration, time and frequency of administration, drug combination(s), reaction sensitivities, and tolerance/response to therapy.

The polypeptide may be delivered at or around menstruation. Depending upon the pharmaceutical composition, the invention may be delivered over a variety of time periods (e.g. several times per day, daily, alternate days or weekly) depending on half-life and clearance rate of the particular formulation. The total duration of therapy may be single dose, or last for days or weeks with multiple doses administered. For example, a 400-500 mg dose can be administered orally twice daily for 5-7 days, or a 1-2 g single dose can be administered orally. Alternatively, 5 g of a 0.75-2% gel, cream or lotion can be applied topically, preferably in the vagina, 1-2 times daily for 3-7 days, in some embodiments preferably at night. A pessary may also be utilised under similar dosage regimes to those described for gels, creams and lotions described above.

All of the features described herein (including any accompanying claims, abstract and drawings), and/or all of the steps of any method so disclosed, may be combined with any of the above aspects in any combination, except combinations where at least some of such features and/or steps are mutually exclusive. Specifically, any of the active agents and compositions described herein can be used in any of the described methods of treatment. Any and all such combinations are explicitly envisaged as forming part of the invention.

### EXAMPLES

### Example 1. Endolysin Discovery

Polypeptides of the invention were identified using Zeus, a proprietary endolysin discovery software developed by CC Biotech Ltd. Zeus acts as a DNA and amino acid sequence search tool. The Zeus software identified polypeptides of the invention using a focused DNA and amino acid sequence search programmed by the inventors.

### Example 2. Curation

Step 1

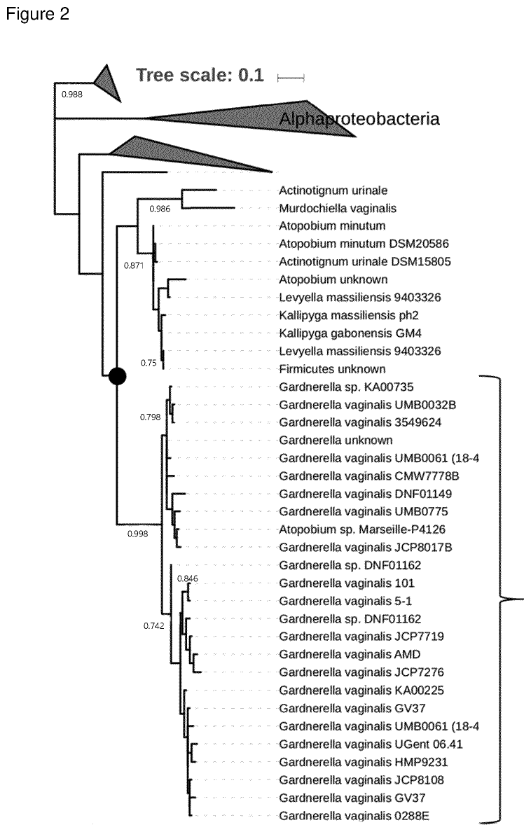

The expected host range of each candidate was inferred using phylogenetic cladistics. The top 50 protein homologues of each candidate were obtained by BLASTp and used to construct phylogenetic trees (FIG. 2). It was important to ensure that selected candidates were specific for G. vaginalis, with activity against healthy vaginal bacteria unlikely. Therefore, endolysins sharing no homologous sequences derived from Lactobacillus spp., Lactobacilli bacteriophage, or other healthy vaginal commensal bacteria were preferentially selected as candidates. Endolysins with homologous sequences described in Lactobacillus spp., Lactobacilli phage, or healthy vaginal commensal bacteria were preferentially selected if G. vaginalis derived endolysins formed distinct and discrete phylogenetic clades away from endolysin homologues derived from healthy commensal bacteria, indicating no/a distant evolutionary history from these lactobacilli endolysins. Further candidates which shared amino acid sequence identity with lactobacilli endolysins were also selected, if preferential conditions couldn't be met. The candidates identified in example 1 and using this process are not likely to target healthy vaginal commensal bacteria, but specifically target and kill G. vaginalis.

Step 2

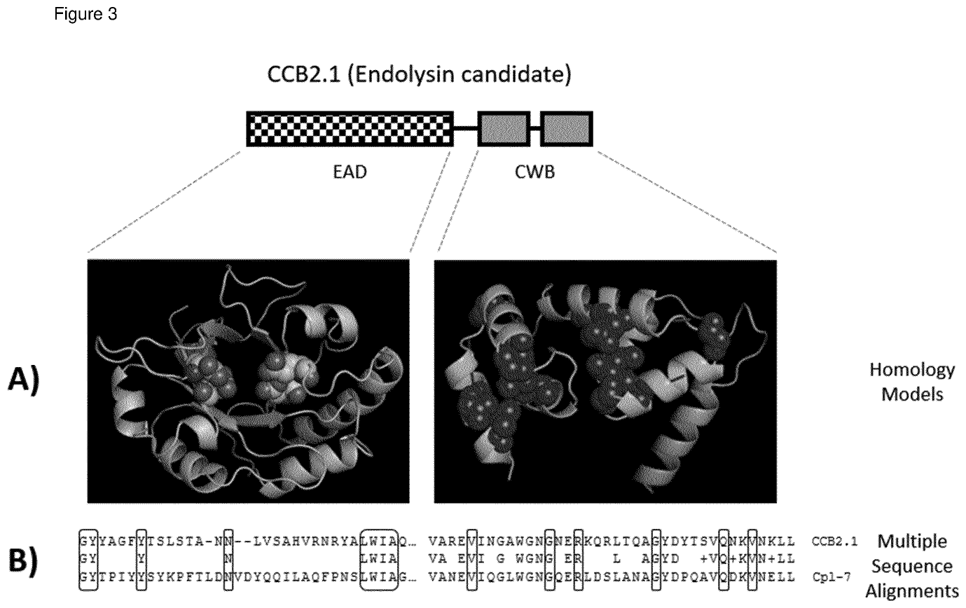

It was important to establish whether mutations rendered endolysin candidates non-functional. To do so, homology models of each candidate endolysin were constructed using crystal structures of similar characterised endolysins as templates. Amino acids implicated in peptidoglycan substrate binding and catalytic activity of the EAD and CWBs were identified in these homology models (FIG. 3A) and multiple sequence alignments were used to further verify these data (FIG. 3B).

This analysis enabled selection of endolysins by the applicants having activity against Gardnerella vaginalis from those containing deleterious mutations: the endolysin sequences lacking critical catalytically active or peptidoglycan substrate binding residues were removed from our reservoir of candidates. This curation step identified 60 functional and selective endolysins, including CCB2.1.

Step 3

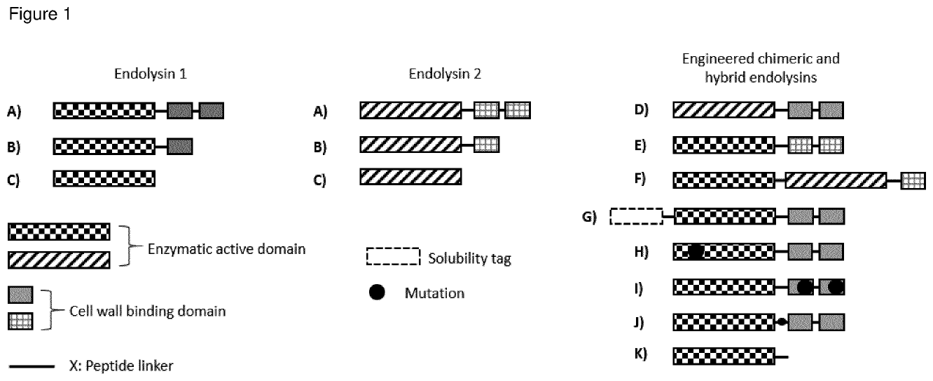

Polypeptides of the invention, discovered as above, were manipulated through addition, truncation, mutation and exchange of amino acid sequences. Mutated polypeptides of the invention, with R1a-Xn domain organisations, were constructed from parent polypeptides of the invention using recombinant DNA technology and synthetic biology technology and were characterised experimentally. Examples include CCB2.4, CCB3.2 and CCB4.2. Mutated polypeptides of the invention with 80, 81.7, 83.6, 84.97, 87.2, 90.2 and 94.8% amino acid sequence similarity to parent polypeptides of the invention were constructed using recombinant DNA technology and synthetic biology technology and were characterised experimentally. Examples include CCB2M80, CCB2M81_7, CCB2M83_6, CCB2M84_97, CCB2M87_2, CCB2M90_2 and CCB2M94.8. Chimeric polypeptides of the invention, with R1a-Xn-R2b domain organisations, were constructed using recombinant DNA technology and synthetic biology technology and were characterised experimentally. Examples include CCB2.2, CCB230a, CCB230b, CCB240, CCB260, CCB270 and CCB280. Shown in part schematically in FIG. 1.

### Example 3. Preliminary Production

Protein Expression Protocol:

Creating Stocks of Each Vector
i) Each vector master stock was resuspended in 15 ul of either double autoclaved DI water, or sterile DNA elution buffer from a qiagen plasmid prep. kit.
ii) Each vector was transformed into E. coli DH5alpha for storage. In brief, the vector containing gene sequences encoding polypeptides of the invention was introduced into E. coli DH5alpha via standard transformation protocols. Transformant colonies were isolated from LB containing kanamycin at a final concentration of 50 ug/ml. Single transformant colonies of Escherichia coli containing pET30a vectors including a gene sequence encoding a polypeptide of the invention were inoculated in falcon tubes containing 10 ml sterile LB broth with a final concentration of 50 ug/ml Kanamycin. Inoculated falcon tubes were incubated overnight (12 h) at 37° C. in a shaking incubator (170 rpm), or to turbidity.
iii) A sample of turbid inoculum was added to 500 ul of autoclave sterilised 50% glycerol and eluted into cryovials for long term storage at −80° C.
iv) pET30a plasmids containing DNA sequences encoding polypeptides of the invention were extracted from the remaining 9.5 ml of turbid inoculum using a Qiagen or similar plasmid purification kit. Plasmid stocks were stored at −20° C. long term.
v) To ensure the each plasmid containing gene sequences encoding polypeptides of the invention was correct the gene sequence encoding the polypeptide of the invention was sequenced use standard primers, T7 (Eurofins 5′ TAA TAC GAC TCA CTA TAG GG 3′) and T7 term (Eurofins 5′ CTA GTT ATT GCT CAG CGG T 3′). Plasmids containing gene sequences encoding polypeptides of the invention were digested using a restriction enzyme creating a bespoke restriction pattern to further validate that the plasmids containing gene sequences encoding polypeptide sequences described in this invention were sequence correct.

Preparing Plates and Growing Gardnerella Vaginalis
i) Columbia Blood agar plates were prepared following manufacturers protocol, G. vaginalis ATCC 14018, G. vaginalis ITM 1044, G. vaginalis ITM 2269, G. vaginalis ITM 2299, and G. vaginalis ITM 3330 were streaked out to single colonies. The bacteria were incubated at 35° C. in anaerobic conditions for 48 h, or until single colonies are identified. 20 square/rectangular Columbia Blood agar plates were prepared for bioassays.
ii) PCR was used to amplify a region of the G. vaginalis 16s rRNA gene from genomic DNA extracted from G. vaginalis strains using standard protocols described in the art. The PCR product was purified and sequenced. The sequenced organisms were restreaked on agar for single colonies and incubated as above once more to maintain viable cultures. This process was repeated prior to antimicrobial bioassays using polypeptides of the invention to validate the identification of the target organism.
iii) A suitable control organism that will not be lysed by candidate endolysins (Escherichia) was grown as above and was used as a negative control in bioassays.
iv) Single G. vaginalis colonies were taken and inoculated in brain heart infusion broth supplemented with glucose. G. vaginalis strains were selected showing best growth. Each G. vaginalis strain was grown to mid-logarithmic phase under anaerobic conditions before transference to sterile falcon tube and pelleting bacteria through gentle centrifugation at 3000×g for 5 minutes, or until supernatant was clear. The supernatant was removed carefully, and the pellet kept on ice.
v) Before use, the G. vaginalis strains were resuspended, and negative control organisms, to 1 McFarlands in buffer 1 (described below).
vi) 200 ul of cell suspension containing individual bacterial strain to be assayed was evenly eluted and spread on to individual square Columbia agar plates for bacterial lawn growth and allowed to dry.

Protein Expression
i) Each gene expression plasmid containing a gene sequence encoding a polypeptide of the invention into was transformed into E. coli BL21(DE3), or another appropriate gene expression host (e.g. E. coli ArcticExpress(DE3). Transformants were incubated overnight at 37° C. on LB agar containing an appropriate concentration of antibiotic for plasmid selection (e.g. for pET30a expression vectors a final concentration of 50 ug/ml of kanamycin was used). Successfully transformed single colonies were picked from LB agar plates containing appropriate antibiotics and inoculated into separate flacon tubes containing 10 ml sterile LB broth supplemented with an appropriate concentration of antibiotic for plasmid selection. This process was repeated using a control organism e.g. E. coli BL21(DE3) with no plasmid on LB agar without kanamycin, and in LB broth containing no kanamycin.
ii) E. coli BL21(DE3) transformed with a gene expression plasmid containing a gene sequence encoding a polypeptide of the invention was incubated at 37° C. until an OD600 of 0.4-0.5 was reached. At which point the falcon tubes were removed from the incubator and placed on ice. Expression of genes encoding a polypeptide sequence of the invention in the expression host was induced via the addition of filter sterilised IPTG to each falcon tube (Other appropriate means of gene expression can be used). Flacon tubes were further incubated under appropriate conditions for recombinant protein production (e.g. 4° C. or 15° C. for 16 or 24 hours, 180 rpm). The same process was repeated using E. coli BL21(DE3) without the plasmid, using LB broth without antibiotics.
iii) An ice cold, and filter sterilised, 20 mM Tris HCl (pH 8.5) sonication buffer containing cOmplete™, Mini, EDTA-free Protease Inhibitor Cocktail, and 20% glycerol (Buffer 1) was prepared. Other appropriate buffers were also used including PBS.
iv) Once incubation at 4° C. or 15° C. for 16 or 24 hours, 180 rpm was complete, falcon tubes were removed from the incubator and the final cell density of each culture was determined by absorbance at 600 nm using a spectrophotometer. All cell densities were normalised through appropriate dilution using sterile LB broth. Next, cell suspensions were centrifuged at 4000×g for 7 minutes, or until cells pelleted. Supernatant was removed. 10 ml of sterile LB was added to cell pellets and pellets were resuspended. This process was repeated a further 3 times. Repeating this cell pellet washing removed all kanamycin, or other appropriate antibiotic used for plasmid selection, from the pelleted cells prior to extraction.
v) Once the supernatant was removed, cell pellets were kept on ice. 1 ml of filter sterilised buffer 1 was added to each tube and the cell pellet was gently suspended. Cell suspensions were transferred to sterile 2 ml Eppendorf tubes, and kept on ice. Cells within the suspension were fully lysed by pulse sonication.
vi) A 100 ul sample of the total cell lysate (TCL) was removed and placed on ice for analysis of recombinant protein production and yield in the heterologous expression host of choice. Analytical techniques include SDS-PAGE and Western blot analysis.

Endolysin candidates can be produced in good yields in the heterologous bacterial host of choice, E. coli BL21(DE3) (FIG. 4) using the methods described. SDS-PAGE and Western blot analysis showed all polypeptide sequences of the invention were produced at high yields as recombinant proteins in the heterologous hosts E. coli BL21(DE3), or E. coli ArcticExpress(DE3), including endolysin polypeptides of the invention CCB2.1, CCB3.1, CCB4.1, CCB6.1, CCB7.1 and CCB8.1, mutated endolysin polypeptides of the invention CCB2.2, CCB2.3, CCB2.4, CCB3.2, CCB4.2, CCB2M80, CCB2M81_7, CCB2M83_6, CCB2M84_97, CCB2M87_2, CCB2M90_2 and CCB2M94.8, and chimeric endolysin polypeptides of the invention CCB230a, CCB230b, CCB240, CCB260, CCB270 and CCB280.
vii) Beyond identification of recombinant polypeptide production and yield, total cellular protein (TCP) can be analysed to determine the solubility characteristics of recombinants polypeptide. To do so, the remaining 0.9 ml of TCP was centrifuged at 20,000×g for 20 minutes, at 4° C., in order to separate soluble and insoluble cellular proteins. The supernatant was removed from the pelleted material and placed in tubes at 4° C. This fraction contains soluble cellular protein. The pelleted material was suspended in 200 μl of buffer 1 and kept on ice, this fraction contains insoluble cellular protein. Both cellular protein fractions were analysed via SDS-PAGE and Western blot to determine the solubility of recombinant polypeptide sequences of the invention.

A subset of the polypeptides of the invention were analysed to determine solubility. CCB2.2, CCB2.4, CCB3.2, CCB4.1, CCB4.2, CCB7.1, CCB8.1, CCB280, CCB270, CCB240, CCB230b, CCB2M94_8, CCB2M90_2 and CCB2M87_2 showed good solubility as recombinant proteins when produced in either E. coli BL21(DE3), or E. coli BL21 ArcticExpress(DE3). The subset of polypeptide sequence analysed included wild type endolysin polypeptide sequences, mutated endolysin polypeptides and chimeric endolysin polypeptides. (FIG. 4w-ax)

All polypeptides of the invention were produced at yields of between 30 and 100 mg/L. Of the candidates, CCB2.1 was most abundant (100 mg/L), while CCB4.2 and CCB2.4 were most soluble (>70% solubility) before optimisation. These results validate our ability to increase endolysin solubility through rational protein engineering: CCB2.4 and CCB4.2 are truncated versions of CCB2.1 and CCB4.1 respectively with a R1a-Xn protein domain architecture. Both CCB2.4 and CCB4.2 lack a C-terminal cell wall binding domain of their respective parent proteins, CCB2.1 and CCB4.1. (FIG. 1b, k). Protein solubility can also be improved by 1) addition of solubility tags (GST, MBP etc.—see FIG. 1g), 2) alteration of gene expression conditions, 3) creation of chimeric endolysin polypeptides of the invention (FIG. 1d,e,f,k), and 4) through introduction of point mutations to endolysin polypeptides of the invention (FIG. 1h, i, j), as well as other mutations and methods. Manipulation of culture conditions, specifically co-expression of bacterial chaperone gene sequences cpn10 and cpn60 in E. coli ArcticExpress(DE3) optimised CCB2.2 recombinant protein production. This optimisation step enabled purification of CCB2.2 to 90% purity>16 mg/L (FIG. 5), exceeding the quantity needed to carry out preliminary downstream bioassays described below.

Constructing chimeric endolysin polypeptides with R1a-Xn-R2b domain architectures from polypeptide sequences of the invention increased recombinant protein solubility. Substitution of a linker and cell wall binding domain from CCB230b with a linker region and cell wall binding domains from CCB7.1 (forming CCB270) increased soluble recombinant protein yield in E. coli BL21(DE3) from 10 mg/L to 18 mg/ml (FIG. 4as, at, aw, ax) Furthermore, introduction of amino acid mutations, specifically amino acid substitutions, increased solubility of endolysin polypeptides of the inventions. Specifically, introduction of R4K, V10E, 136T, H38C, E45Q, A53V, 181M, V134T, D145N, D159N, S172N, D176P, 1178V, V189K, N193S, V1941, Y209D, T210Q, P212A, Q213A, D228A, N237Q, Q241S, T247A, S282N, T283E, D296A amino acid substitutions improved the solubility of CCB2M90_2 from 75 mg/L to 85 mg/L, forming CCB2M94_7 (FIG. 4y, z, aa, ab).

### Example 4. Preliminary Bioactivity

E. coli BL21(DE3) cell lysates were used to demonstrate whether recombinant G. vaginalis derived endolysins including CCB2.1, CCB2.2, CCB2.3, CCB2.4, CCB3.2, CCB4.1, CCB4.2, CCB7.1, CCB8.1 and endolysins with N-terminal modifications, specifically hexa-histidine and Tobacco Etch Virus protease cleavage sites, displayed anti-Gardnerella spp. activity. These experiments represent an adaptation of a standard zone of inhibition microbiological bioassay. E. coli BL21(D3) transformed with expression vectors containing endolysin gene sequences encoding polypeptides of the invention were cultured as described in Example 3. Expression of genes encoding endolysin polypeptides of the invention was achieved in an appropriate recombinant expression host (e.g. E. coli BL21(DE3)) as described in Example 3. Normalised total cell lysates containing recombinant endolysin polypeptides of the invention were prepared as described in example 3. Negative control organisms, specifically heterologous host organisms transformed with an empty gene expression vector (e.g. E. coli BL21(DE3) containing pET30a) were cultured as described in example 3. Cell lysate of control organisms were prepared as described in example 3.

Cell lysates containing recombinant polypeptides of the invention, cell lysates from negative control organisms and protein extraction buffers (e.g. buffer 1) were added to wells cut into Columbia blood agar plates, brain heart infusion agar supplemented glucose, or other appropriate media, overlaid with a dried suspension of Gardnerella spp., specifically G. vaginalis ATCC 14018, prepared as described in example 3, and incubated anaerobically at 35° C. for 12, 24, 48 and 72 hours. The zone of Gardnerella spp. growth inhibition after incubation anaerobically at 35° C. for 12, 24, 48 and 72 hours were recorded for all samples. All the endolysins tested (including CCB2.1, CCB2.2, CCB2.3, CCB2.4, CCB3.2, CCB4.1, CCB4.2, CCB7.1, CCB8.1 and endolysins with N-terminal modifications) showed anti-Gardnerella spp. bioactivity (FIG. 6) clearly killing, and preventing growth of, G. vaginalis ATCC 14018 cells overlaid on the agar plates proximal to the wells (FIG. 6). Zones of inhibition ranged from 6.5 mm to 16 mm in size (FIG. 6b). Cell lysates extracted from negative control organisms (e.g. E. coli BL21(DE3) containing pET30a) showed no anti-Gardnerella spp bioactivity, nor did cell lysate buffers (Buffer 1), demonstrating the anti-Gardnerella bioactivity to be attributed to the recombinant endolysin polypeptides of the invention. No zones of inhibition were observed for any controls: Zone of inhibition size was 0 mm (FIG. 6b). This work demonstrates anti-Gardnerella endolysin activity for the first time with remarkable potency. Each experiment comprised two technical replicates and three biological replicates. Although soluble recombinant CCB2.1 concentrations within cell lysate were low (data not shown), the small quantities of endolysin present inhibited growth of Gardnerella vaginalis effectively when total recombinant protein was normalised.

### Example 5. Antibiotic Resistance Profile

60% of clinical isolates of G. vaginalis show resistance to metronidazole. Our data supports this: when G. vaginalis was exposed to 2.5 mg/ml and 5 mg/ml (1000-fold higher than reported minimum inhibitory concentrations) of metronidazole>50 G. vaginalis colonies were observed within the zone of metronidazole growth inhibition. The ability of G. vaginalis to grow in the presence of metronidazole at described concentrations indicates these mutants to be resistant to the antibiotic metronidazole. Conversely, no spontaneously endolysin resistant G. vaginalis mutants were observed when repeatedly grown in the presence of CCB2.1 lysate (FIG. 7), suggesting that the resistance profile of endolysin candidates may be lower than that of the existing front line clinically utilised therapeutic, metronidazole.

### Example 6. Selectivity of Anti-Gardnerella Spp. Bioactive Endolysin Candidates

Endolysin polypeptides have selective antimicrobial activity at the strain, species and genus level, and therefore can be used as precision antibacterials. We demonstrated polypeptides of the invention to have selective antimicrobial activity: specifically, the ability of multiple endolysin candidates to kill and prevent the growth of Gardnerella spp., but to have no effect on the growth of bacteria found in the healthy vagina and organisms known to have beneficial properties within the vaginal microbiome, or flora including organisms of the Lactobacillus spp. Zone of inhibition assays were utilised as described in example 4 to determine selectivity of endolysin polypeptides of the invention. Materials were prepared as described in examples 3 and 4.

Lactobacillus gasseri CCUG 31451 T, Lactobacillus crispatus CCUG 30722T and Lactobacillus jensenii CCUG 21961T were prepared for zone of inhibition assays as follows. Lactobacillus gasseri CCUG 31451T, Lactobacillus crispatus CCUG 30722T and Lactobacillus jensenii CCUG 21961T were individually struck on De Man, Rogosa and Sharpe (MRS) agar and incubated anaerobically at 35° C. for 24 hours. Single colonies of each Lactobacillus spp. were individually inoculated into sterile MRS broth and incubated anaerobically at 35° C. until cultures reached mid-logarithmic growth phase. The turbidity of each culture was adjusted to a turbidity of 1 McFarland, and each cell suspension was evenly eluted and spread on to an MRS agar plate. Wells were cut to facilitate zone of inhibition testing.

Cell lysates containing recombinant polypeptides of the invention, cell lysates from negative control organisms (e.g. E. coli BL21(DE3) transformed with pET30a) and protein extraction buffers (e.g. buffer 1), were added to wells cut into MRS agar plates overlaid with Lactobacillus gasseri CCUG 31451T, Lactobacillus crispatus CCUG 30722T and Lactobacillus jensenii CCUG 21961T, respectively. Similarly, cell lysates containing recombinant polypeptides of the invention, cell lysates from negative control organisms (e.g. E. coli BL21(DE3) transformed with pET30a) and protein extraction buffers (e.g. buffer 1), were added to wells cut into BHI (supplemented with glucose) agar plates overlaid with G. vaginalis ATCC 14018. All plates were incubated anaerobically for 12, 24, 48 and 72 hours at 35° C. Zones of inhibition were measured and recorded.

No growth retardation, growth prevention or evidence of antibacterial activity caused by cell lysate containing endolysin polypeptide of the invention towards Lactobacillus gasseri CCUG 31451T, Lactobacillus crispatus CCUG 30722T or Lactobacillus jensenii CCUG 21961T was observed. Specifically, the zone of inhibition size was 0 mm for wells containing cell lysates containing CCB2.1 and CCB2.4 (FIG. 8). Conversely, and in agreement with Example 4, cell lysates containing CCB2.1 and CCB2.4 showed antibacterial activity towards G. vaginalis ATCC 14018: average zones of inhibition were 13.125 mm and 9.5 mm, respectively (FIG. 8). No zones of inhibition were observed for any controls. Each experiment comprised two technical replicates and three biological replicates.

This work demonstrated the selective antimicrobial bioactivity of endolysin polypeptides of the invention towards Gardnerella spp., but with no antimicrobial against other organisms found within the vaginal microbiome, specifically organisms of the Lactobacillus spp.

1-29. (canceled)

30. A polypeptide having anti-Gardnerella spp. activity comprising a hydrolase domain, wherein the hydrolase domain comprises or consists of an amino acid sequence selected from any one of SEQ ID NOs: 5, 3, 6, 2, 4, or 1, or a sequence having at least 80, 85, 90, 95, 96, 97, 98, 99, 99.5 or 100% identity thereto or a fragment thereof

31. A polypeptide of claim 30, wherein the polypeptide comprises or consists of an amino acid sequence selected from:
(i) SEQ ID NO. 5, SEQ ID NO. 12, SEQ ID NO.19, SEQ ID NO.26, and SEQ ID NO.61;
(ii) SEQ ID NO. 3, SEQ ID NO. 10, SEQ ID NO.17, SEQ ID NO.23, SEQ ID NO.54-60, and SEQ ID NO.70;
(iii) SEQ ID NO. 6, SEQ ID NO. 13, SEQ ID NO.20, and SEQ ID NO.26;
(iv) SEQ ID NO. 2, SEQ ID NO. 9, SEQ ID NO.16, SEQ ID NO. 22, SEQ ID NO.51-53, and SEQ ID NO.76; or (v) SEQ ID NO. 4, SEQ ID NO. 11, SEQ ID NO.18, and SEQ ID NO.24.

32. The polypeptide of claim 30, having the formula R1a-Xn, where R1 is a hydrolase domain, X is a linker, a is an integer between 1 and 3, and n is an integer from 1 to 10.

33. The polypeptide of claim 30 comprising a cell wall binding domain, optionally wherein the cell wall binding domain binds specifically to Gardnerella spp. bacteria.

34. The polypeptide of claim 33, wherein the cell wall binding domain comprises a CW_7, SH3b, COG5263, SPOR, LysM, PG_Binding_1, PhageMin_tale, Caudovirus_tape_meas_N-terminal, TrusA_like, and/or MFS domain.

35. The polypeptide of claim 33, wherein the cell wall binding domain comprises or consists of an amino acid sequence selected from any one of SEQ ID NOs: 7 to 13 or a sequence having at least 80, 85, 90, 95, 96, 97, 98, 99, 99.5 or 100% identity thereto or a fragment thereof.

36. The polypeptide of claim 33 comprising two cell wall binding domains.

37. The polypeptide of claim 33, wherein one or more cell wall binding domain is C-terminal to the hydrolase domain, or wherein one or more cell wall binding domain is N-terminal to the hydrolase domain.

38. The polypeptide of claim 33 having the formula:
(a) R1a-Xn-R2b, where R1 is a hydrolase domain, X is a linker, R2 is a cell wall binding domain, a is an integer between 1 and 3, b is an integer between 1 and 10, preferably 1 or 2, and n is an integer from 1 to 10; or
(b) R1a-Xn-R2b-Ym-R3c, where R1 is a hydrolase domain, X and Y is a linker, R2 is a cell wall binding domain, R3 is a hydrolase domain, a is an integer between 1 and 3, b is an integer between 1 and 10, preferably 1 or 2, c is an integer between 1 and 10, and n and m is an integer from 1 to 10.

39. A polypeptide comprising an amino acid sequence selected from any one of SEQ ID NOs: 1 to 13 or 21 to 26 or a sequence having at least 80, 85, 90, 95, 96, 97, 98, 99, 99.5 or 100% identity thereto or a fragment thereof, or a combination thereof; preferably wherein the sequence is an amino acid sequence selected from any one of SEQ ID NO: 2-6 or a sequence having at least 80, 85, 90, 95, 96, 97, 98, 99, 99.5 or 100% identity thereto or a fragment thereof.

40. The polypeptide of claim 39, further comprising an amino acid sequence selected from any one of SEQ ID NOs: 14 to 20 or a sequence having at least 80, 85, 90, 95, 96, 97, 98, 99, 99.5 or 100% identity thereto or a fragment thereof; preferably wherein the sequence is an amino acid sequence selected from any one of SEQ ID NO: 16-13 or a sequence having at least 80, 85, 90, 95, 96, 97, 98, 99, 99.5 or 100% identity thereto or a fragment thereof.

41. The polypeptide of claim 30 comprising or consisting of any one of SEQ ID NO: 21-77, or a sequence having at least 80, 85, 90, 95, 96, 97, 98, 99, 99.5 or 100% identity thereto or a fragment thereof, and optionally comprising or consisting of any one of, SEQ ID No: 21, 23, 25, 26, 73, 74. 75, 76 and 77 or a sequence having at least 80, 85, 90, 95, 96, 97, 98, 99, 99.5 or 100% amino acid sequence identity thereto, or a fragment thereof, and further optionally comprising or consisting of any one of, SEQ ID No: 23, 25, 26, 76 and 77 or a sequence having at least 80, 85, 90, 95, 96, 97, 98, 99, 99.5 or 100% amino acid sequence identity thereto, or a fragment thereof.

42. An isolated nucleic acid encoding the polypeptide of claim 30.

43. A vector comprising the isolated nucleic acid of claim 42.

44. A cell comprising the nucleic acid of claim 42 or a vector comprising it.

45. A composition comprising the polypeptide of claim 30, an isolated nucleic acid encoding the polypeptide of claim 30, a vector comprising the isolated nucleic acid encoding the polypeptide of claim 30, or a cell comprising the isolated nucleic acid encoding the polypeptide of claim 30 or a vector comprising the isolated nucleic acid encoding the polypeptide of claim 30.

46. The composition of claim 45, wherein the composition is a pharmaceutical composition comprising one or more pharmaceutically acceptable excipient.

47. The composition of claim 45 for vaginal administration, optionally in the form of a cream, gel, lotion, tampon or pessary.

48. A method of treating or preventing a bacterial infection in a subject, the method comprising administering to the subject a therapeutically effective amount of a composition of claim 45.

49. A method according to claim 48, wherein the polypeptide, nucleic acid, vector, cell or composition prevent, disperse, treat and/or decolonize a bacterial biofilm and/or kill or prevent growth of a bacteria and optionally wherein the bacteria include Gardnerella spp., preferably wherein the bacteria include one or more of G. leopoldii, G. piotii, and G. swidsinskii and G. vaginalis

50. A method according to claim 48, wherein the bacterial infection is bacterial vaginosis.
